(19) United States
(12) Patent Application Publication
(10) Pub. No.: US 2022/0220135 A1
(43) Pub. Date: Jul. 14, 2022

(54) HIGHLY SELECTIVE AND POTENT NPP1 INHIBITORS BASED ON URIDINE-5'-P,-DITHIOPHOSPHATE ANALOGUES

(71) Applicants: BAR-ILAN UNIVERSITY, Ramat-Gan (IL); ICHILOV TECH LTD, Tel-Aviv (IL); LAVAL UNIVERSITY, Quebec (CA); UNIVERSITY OF BONN, Bonn (DE)

(72) Inventors: Hana Bilha FISCHER, Shoham (IL); Molhm NASSIR, Tira (IL); Vadim ZELIKMAN, Rishon Lezion (IL); Uri Moshe ARAD, Tel-Aviv (IL); Jean SEVIGNY, Quebec (CA); Christa MULLER, Bonn (DE)

(73) Assignees: BAR-ILAN UNIVERSITY, Ramat-Gan (IL); ICHILOV TECH LTD, Tel-Aviv (IL); LAVAL UNIVERSITY, Quebec, QC (CA); UNIVERSITY OF BONN, BONN (DE)

(21) Appl. No.: 17/604,698

(22) PCT Filed: Apr. 18, 2020
(86) PCT No.: PCT/IL2020/050456

§ 371 Date: Oct. 18, 2021

Related U.S. Application Data
(63) Continuation of application No. 62/835,914, filed on Apr. 18, 2019.

(51) Int. Cl. C07F 9/6561; C07H 19/20

(52) U.S. Cl. CPC C07F 9/6561; C07H 19/20

(57) ABSTRACT

The invention relates to a novel highly selective and potent NPP1 inhibitors, compositions comprising such, and their use as pharmaceuticals.

### FIELD OF THE INVENTION

The invention relates to a novel highly selective and potent NPP1 inhibitors, compositions comprising such, and their use as pharmaceuticals.

### BACKGROUND OF THE, INVENTION

Ectonucleotide pyrophosphatase/phosphodiesterase-1 (NPP1) is a membrane glycoprotein with Zn(II)-binding extracellular catalytic-site. NPP1 hydrolyzes bonds of nucleotides such as ATP. Hydrolysis of ATP by NPP1 results mainly in the formation of AMP and pyrophosphate (PPᵢ), and also ADP and inorganic phosphate (Pi). It is noteworthy that PPᵢ can be further converted to Pᵢ by alkaline phosphatase. AMP produced by NPP1 can be hydrolyzed by 5′-nucleotidase (5′-NT, CD73) or alkaline phosphatase to adenosine.

An abnormally high level of NPP1 is found in calcific aortic valve disease (CAVD), which is the most frequent heart valve disorder. Overexpression of NPP1 leads to increased mineralization of the aortic valve and to valvular interstitial cell apoptosis. NPP1 is also a major contributor to the pathological NPPase activity and elevated PPi levels observed in a joint pathology—Calcium Pyrophosphate Dihydrate (CPPD) disease. In addition to NPP1, the phylogenetically related enzyme, NPP3, also catalyzes the hydrolysis of phosphodiester bonds. Yet, while excess activity of NPP1 results in health disorders such as CAVD or CPPD, excess activity of NPP3 triggers an allergic response.

Currently, there are no known therapies to slow down CAVD progression, making surgical valve replacement as the only effective treatment for aortic stenosis. Likewise, today there are no CPPD disease modifying drugs, and the only available treatments are symptomatic. Hence, a promising avenue for a novel pharmacological treatment for either CAVD or CPPD could be selective NPP1 inhibitors.

Previously, biocompatible, water-soluble, and selective NPP1 inhibitors, which do not affect other ectonucleotidases such as ecto-nucleoside triphosphate diphosphohydrolase (NTPDases) and ecto-5′-ectonucleotidase (5′-NT), and that do not interfere with nucleotide receptors, P2-Rs, activation, were reported. Destipe certain advantageous properties, said inhibitors have not displayed desired properties required for successful therapeutics. Thus, developing potent, selective, biocompatible, and stable inhibitors of NPP1 still represents an unmet medical need.

### SUMMARY OF THE INVENTION

The present invention provides Highly Selective and Potent NPP1 Inhibitors Based on Uridine-5′-Pα,α-dithiophosphate Analogues having the the structure:

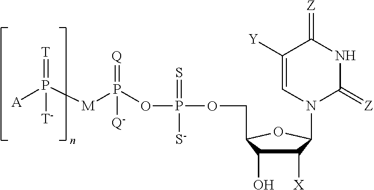

or a pharmaceutically acceptable salt thereof, wherein:

X is H, OH or NH₂; Z is O, S, or Se; Y is H, OR, SR, NHR; R is straight or branched, optionally substituted alkyl or cycloalkyl; Q is O or S; M is O, OH, CH₂, CCl₂, CBr₂, or CF₂; T is O or S; A is O—, OH, or a nucleoside; and n is 0 or 1.

The present invention yet further provides a pharmaceutical composition comprising a Compound having the structure,

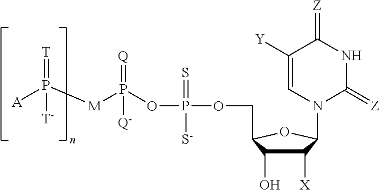

or a pharmaceutically acceptable salt thereof, wherein:

X is H, OH or NH₂; Z is O, S, or Se; Y is H, OR, SR, NHR; R is straight or branched, optionally substituted alkyl or cycloalkyl; Q is O or S; M is O, OH, CH₂, CCl₂, CBr₂, or CF₂; T is O or S; A is O— or a nucleoside; n is 0 or 1; and at least one pharmaceutically acceptable carrier.

The invention yet further provides a method for treating a condition associated with elevated levels of NPP1 in a subject in need of such treatment comprising administering to the subject a therapeutically effective amount of the Compound having the structure:

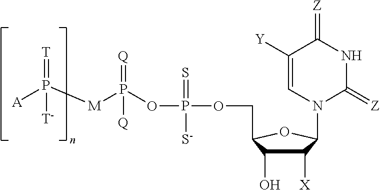

or a pharmaceutically acceptable salt thereof, wherein:

X is H, OH or NH₂; Z is O, S, or Se; Y is H, OR, SR, NHR; R is straight or branched, optionally substituted alkyl or cycloalkyl; Q is O or S; M is O, OH, CH₂, CCl₂, CBr₂, or CF₂; T is O or S; A is O— or a nucleoside;

The invention yet further provides a process for the manufacture of a Compound having the structure:

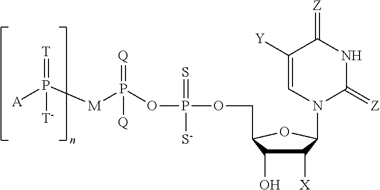

a) Treating Uridine-2′,3′-O-methoxymethylidene, with 1,3,2-dithiaphospholane to obtain a compound of Formula I;

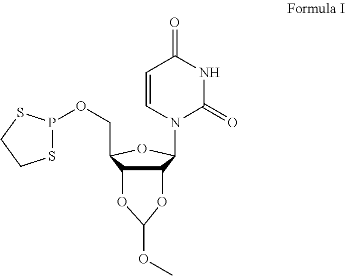

b) oxidizing the compound of formula I by powdered elemental sulfur at room temperature to obtain a compound of Formula II;

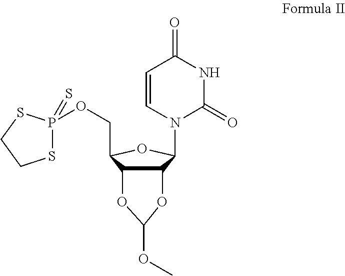

c) Subjecting the compound of Formula II to DBU-mediated nucleophilic dithiaphospholane ring-opening;

to thereby manufacture the Compound of the invention.

The invention yet further provides a process for the manufacture of a Compound having the structure:

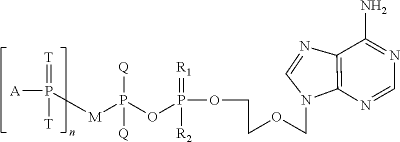

or a pharmaceutically acceptable salt thereof, wherein:

R₁ and R₂ is each independently O or S; Q is O or S; M is O, OH, CH₂, CCl₂, CBr₂, or CF₂; T is O or S; A is O— or a nucleoside; and n is 0 or 1; and at least one pharmaceutically acceptable carrier.

The invention further provides a pharmaceutical composition comprising a Compound having the structure,

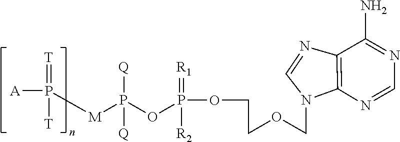

or a pharmaceutically acceptable salt thereof, wherein:

R₁ and R₂ is each independently O or S; Q is O or S; M is O, OH, CH₂, CCl₂, CBr₂, or CF₂; T is O or S; A is O— or a nucleoside; and n is 0 or 1; and at least one pharmaceutically acceptable carrier.

The invention yet further provides a pharmaceutical composition comprising a Compound having the structure:

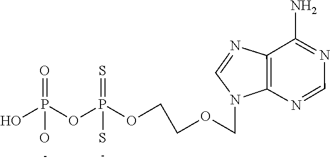 [SMILES: Nc1ncnc2c1ncn2COCCOP(=S)(S)OP(=O)(O)O]

or a pharmaceutically acceptable salt, and at least one pharmaceutically acceptable carrier.

The invention yet further provides a pharmaceutical composition comprising a Compound having the structure:

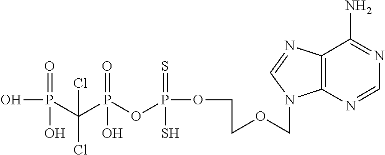 [SMILES: Nc1ncnc2c1ncn2COCCOP(=S)(S)OP(=O)(O)C(Cl)(Cl)P(=O)(O)O]

or a pharmaceutically acceptable salt, and at least one pharmaceutically acceptable carrier.

The invention provides A pharmaceutical composition comprising a Compound having the structure:

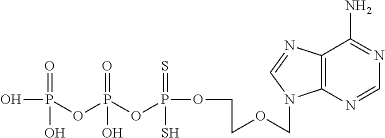 [SMILES: Nc1ncnc2c1ncn2COCCOP(=S)(S)OP(=O)(O)OP(=O)(O)O]

or a pharmaceutically acceptable salt, and at least one pharmaceutically acceptable carrier.

The invention also provides a pharmaceutical composition comprising a Compound having the structure:

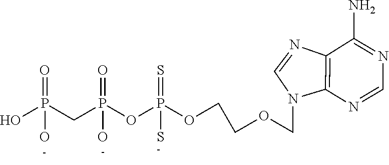 [SMILES: Nc1ncnc2c1ncn2COCCOP(=S)(S)OP(=O)(O)CP(=O)(O)O]

or a pharmaceutically acceptable salt, and at least one pharmaceutically acceptable carrier.

The invention further provides method for treating a condition associated with enhanced NPP1 activity in a subject in need of such treatment comprising administering to the subject a therapeutically effective amount of the pharmaceutical composition of the invention.

The invention yet further provides a method for treating a condition associated with elevated NPP1 levels in a subject in need of such treatment, comprising administering to the subject a therapeutically effective amount of the Compound of the invention.

### BRIEF DESCRIPTION OF THE DRAWINGS

FIG. 1: A. Chemical structure of previously described inhibitors of NPP1 inhibitors; B. Novel selective inhibitors of NPP1;

FIG. 2: Inhibition of NTPDases and NPPs by analogues 9-12 using as substrates ATP (100 μM) for NTPDase1,2,3,8 and pNP-TMP (100 μM) for NPP1,3 (Panel A); and ATP (100 μM) was used as the substrate for NPP1 (Panel B);

FIG. 3: P2Y₆R agonist activity of analogues 9-12 vs. UDP determined by measuring the production of inositol-1-phosphate (IP1);

FIG. 4: 3D (left) and 2D (right) representation of Binding modes of analogues 9-12 at the human NPP1 homology model. (A-B) AMP-like and non-AMP-like binding mode of analogue 9, respectively. (C-D) AMP-like and non-AMP-like binding mode of analogue 10, respectively. (E-F) AMP-like and non-AMP-like binding mode of analogue 11, respectively. (G) Binding mode of analogue 12. The two Zn ions are shown as purple spheres, and the ligands are colored according to atom type (nitrogen atoms are colored in blue; oxygen atoms are colored in red, carbon atoms are colored in gray, and phosphorus atoms are colored in orange). Salt bridges, H-bonds and n-interactions are shown in rainbow, purple and green, respectively;

FIG. 5: Kinetic profiles showing the changes in the percentage of 10 (A); UTP (B); UDP (C), under acidic conditions, pD 2.1 (pH 1.7), as monitored by ₃₁P-NMR at 243 MHz, at 298 K for 23 days;

FIG. 6: Kinetic profile showing the changes in the percentage of 10 under basic conditions, pD 12.1 (pH 11.7), as monitored by ₃₁P-NMR at 243 MHz, at 298 K for 25 days;

FIG. 7: Additional NPP1 inhibitor analogues;

FIG. 8: Concentration-inhibition curve of 22 at human NPP1 vs. 100 μM p-Nph-5′-TMP as a substrate (Ki=16.3±2.5 μM) and Lineweaver-Burk plot of NPP1 inhibition by 22. S, substrate concentration of p-Nph-5′-TMP (μM), v, velocity of enzyme (nmol/min/mg protein); concentration of 22: green circle, 0 μM; blue triangle, 7.5 μM; violet triangle, 30 μM. Each experiment was performed in triplicates;

FIG. 9: Concentration-inhibition curve of 22 at human NPP1 vs. 100 μM ATP as a substrate (Ki=9.60±2.84 μM) and Lineweaver-Burk plot of NPP1 inhibition by 22. S, substrate concentration of ATP (μM), v, velocity of enzyme (nmol/min/mg protein); concentration of 22: green circle, 0 μM; blue triangle, 50 μM; violet triangle, 150 μM. Each experiment was performed in triplicates;

FIG. 10: Evaluation of the ability of analogs 20-28 to inhibit NPPase activity in human chondrocytes. NPPase activity was assayed by measuring the hydrolysis of the chromogenic substrate, p-Nph-5′-TMP. Analogs 20-28 and the natural substrate, ATP, were added at equimolar concentrations (100 μM). All values related to untreated human chondrocytes. (b) Human chondrocytes were incubated with or without analogs 20-28 and assayed for alkaline phosphatase activity by hydrolysis of p-nitrophenyl phosphate;

FIG. 11: Analogs 20-28 are not toxic to primary human chondrocytes. Chondrocytes were incubated with analogs 20-28 at the indicated concentrations for 24 h, and then cell viability was assessed by the XTT assay;

FIG. 12: Second generation of NPP1 inhibitors;

FIG. 13: Concentration-dependent inhibition curves for different inhibitors vs. p-Nph-5′-TMP and ATP as substrate. Figures represent mean±SD of triplicate investigation;

FIG. 14: Inhibition type of analog 7 vs p-Nph-5′-TMP and ATP. data generated by lineweaver-burk plot. α value of ˜1 shows non-competitive inhibition; and

FIG. 15: The effects of analogs 106-109 on primary human chondrocytes. (A) Analogs 106-109 inhibit NPPase activity in primary chondrocytes. NPPase activity was assayed by measuring the hydrolysis of the chromogenic substrate, pnp-TMP. (B) Analogs 106-109 do not substantially inhibit TNAP activity in human derived primary chondrocytes at a concentration of 0.1 mM. Alkaline phosphatase activity was assayed by hydrolysis of p-nitrophenyl phosphate. (C) Analogs 106-109 are not toxic to cultured chondrocytes at a concentration of 0.1 mM. Cell viability was assessed by the XTT assay. Mean (columns) and SD (error bars) of triplicate cell culture experiments are shown.

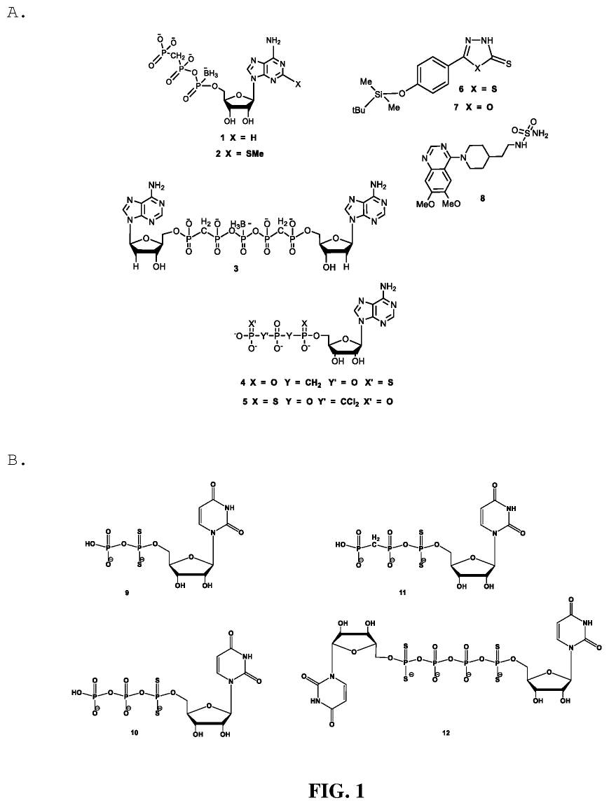

### DETAILED DESCRIPTION OF THE INVENTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The present invention provides a novel inhibitor of NPP1 having the the structure:

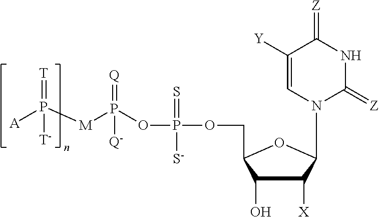

or a pharmaceutically acceptable salt thereof, wherein X is selected from H, OH and NH₂; Z is selected from O, S, and Se; Y is selected from H, OR, SR, and NHR; R is straight or branched, optionally substituted alkyl or cycloalkyl; Q is selected from O and S; M is selected from O, OH, CH₂, CCl₂, CBr₂, and CF₂; T is selected from O and S; A is O—, OH or a nucleoside; and n is 0 or 1. In one embodiment, X is OH; Y is H; Z is O; Q is O; M is CH₂; T is O; n is 1; and A is OH. In another embodiment, X is OH; Y is H; Z is O; Q is O; M is O; T is O; n is 1; and A is OH. In yet another embodiment, X is OH; Y is H; Z is O; Q is O; M is O; T is O; n is 1; and A is a nucleoside. In further embodiment of the invention, X is OH; Y is H; Z is O; Q is S; M is O is H; and n is 0.

The invention further provides a pharmaceutical composition comprising a Compound having the structure,

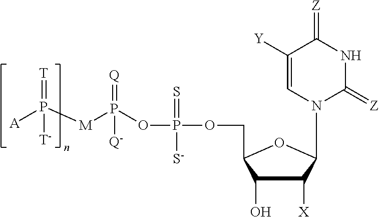

or a pharmaceutically acceptable salt thereof, wherein X is selected from H, OH and NH₂; Z is selected from O, S, and Se; Y is selected from H, OR, SR, and NHR; R is straight or branched, optionally substituted alkyl or cycloalkyl; Q is selected from O and S; M is selected from O, OH, CH₂, CCl₂, CBr₂, and CF₂; T is selected from O and S; A is selected from O—, OH and a nucleoside; and n is 0 or 1; and at least one pharmaceutically acceptable carrier. In one embodiment of the invention, X is OH; Y is H; Z is O; Q is O; M is CH₂; T is O; and N is OH. In another embodiment, X is OH; Y is H; Z is O; Q is O; M is O; T is O; and N is OH. In yet another embodiment, X is OH; Y is H; Z is O; Q is O; M is O; T is O; and N is a nucleoside. In yet further embodiment, X is OH; Y is H; Z is O; Q is S; M is O; T is H; and n is 0.

The invention further provides a method of treating a condition associated with elevated NPP1 levels in a subject in need of such treatment. The subject in need is treated with a therapeutically effective amount of a Compound having the structure:

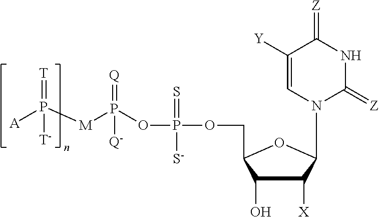

or a pharmaceutically acceptable salt thereof, or with a pharmaceutical composition comprising the Compound and at least one pharmaceutically acceptable carrier. In an embodiment of the invention X is H, OH or NH₂; Z is O, S, or Se; Y is H, OR, SR, NHR; R is straight or branched, optionally substituted alkyl or cycloalkyl; Q is O or S; M is O, OH, CH₂, CCl₂, CBr₂, or CF₂; T is O or S; A is O— or a nucleoside; and n is 0 or 1. In one embodiment, X is OH; Y is H; Z is O; Q is O; M is CH₂; T is O; and N is OH. In another embodiment, X is OH; Y is H; Z is O; Q is O; M is O; T is O; and N is OH. In yet another embodiment, X is OH; Y is H; Z is O; Q is O; M is O; T is O; and N is a nucleoside. In yet further embodiment, X is OH; Y is H; Z is O; Q is S; M is O is H; and n is 0. In another embodiment of the invention, the condition associated with elevated levels of NPP1 is calcific aortic valve disease (CAVD). In further embodiment, the condition associated with elevated levels of NPP1 is Calcium Pyrophosphate Dihydrate (CPPD) disease.

In some embodiments, the invention provides A process for the manufacture of a Compound having the the structure:

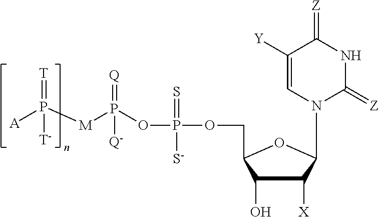

or a pharmaceutically acceptable salt thereof comprising:
a) Treating Uridine-2′,3′-O-methoxymethylidene, with 1,3,2-dithiaphospholane to obtain a compound of Formula I;

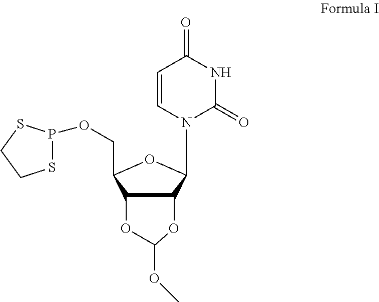

b) oxidizing the compound of formula I by powdered elemental sulfur at room temperature to obtain a compound of Formula II;

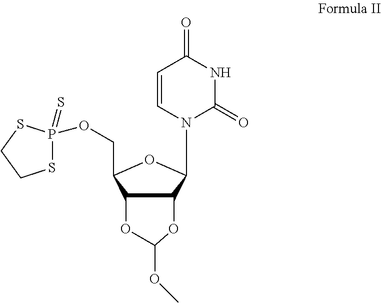

c) Subjecting the compound of Formula II to DBU-mediated nucleophilic dithiaphospholane ring-opening;

to thereby manufacture the Compound.

In some embodiments of the invention, provided a Compound having the structure:

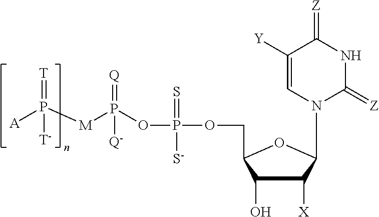

or a pharmaceutically acceptable salt thereof, for use as a medicament, wherein X is selected from H, OH and NH₂; Z is selected from O, S, and Se; Y is selected from H, OR, SR, and NHR; R is straight or branched, optionally substituted alkyl or cycloalkyl; Q is selected from O and S; M is selected from O, OH, CH₂, CCl₂, CBr₂, and CF₂; T is selected from O and S; A is O—, OH and a nucleoside; and, n is 0 or 1. In one embodiment, X is OH; Y is H; Z is O; Q is O; M is CH₂; T is O; and N is OH. In another embodiment, X is OH; Y is H; Z is O; Q is O; M is O; T is O; and N is OH. In yet another embodiment, X is OH; Y is H; Z is O; Q is O; M is O; T is O; and N is a nucleoside. In yet further embodiment, X is OH; Y is H; Z is O; Q is S; M is O is H; and n is 0. In some embodiment, the medicament is used in the treatment of a condition associated with elevated levels of NPP1. In one embodiment of the invention, the condition associated with elevated levels of NPP1 is calcific aortic valve disease (CAVD). In further embodiment, the condition associated with elevated levels of NPP1 is Calcium Pyrophosphate Dihydrate (CPPD) disease.

In some embodiments of the invention, provided a Compound having the structure,

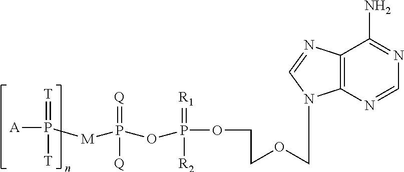

or a pharmaceutically acceptable salt thereof, wherein:

R₁ and R₂ is each independently selected from O and S; Q is selected from O and S; M is selected from O, OH, CH₂, CCl₂, CBr₂, and CF₂; T is selected from O and S; A is selected from O—, OH and a nucleoside; and n is 0 or 1. In one embodiment both R₁ and R₂ are S; Q is O; M is CH₂; T is O; n is 1; and A is O. In yet another embodiment, both R₁ and R₂ are S; Q is O; M is O; T is O; n is 1; and A is OH. In one embodiment, both R₁ and R₂ are S; Q is O; M is O; T is O; and n is 0. In yet another embodiment, both R₁ and R₂ are S; Q is O; M is CCl₂; T is O; and n is 1; and A is O. In one embodiment, provided a compound having the structure:

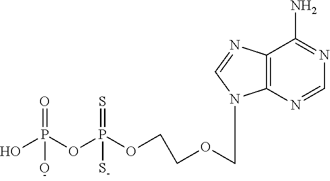

In one embodiment, provided a compound having the structure:

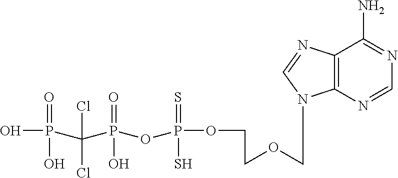 [SMILES: Nc1ncnc2c1ncn2COCCOP(=S)(S)OP(=O)(O)C(Cl)(Cl)P(=O)(O)O]

In one embodiment, provided a compound having the structure:

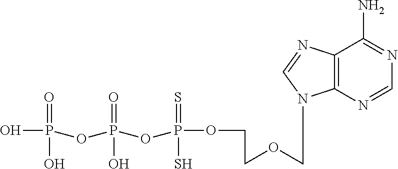 [SMILES: Nc1ncnc2c1ncn2COCCOP(=S)(S)OP(=O)(O)OP(=O)(O)O]

In one embodiment, provided a compound having the structure:

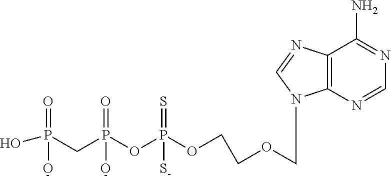

In one embodiment, provided a pharmaceutical composition comprising a Compound having the structure,

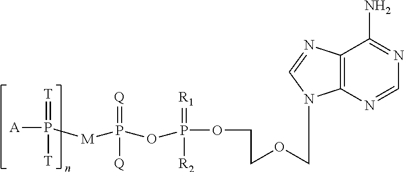

or a pharmaceutically acceptable salt thereof, wherein:

R₁ and R₂ is each independently selected from O and S; Q is selected from O and S; M is selected from O, OH, CH₂, CCl₂, CBr₂, and CF₂; T is selected from O and S; A is selected from O—, OH and a nucleoside; and n is 0 or 1; and at least one pharmaceutically acceptable carrier. In one embodiment, both R₁ and R₂ are S; Q is O; M is CH₂; T is O; n is 1; and A is O. In yet another embodiment, both R₁ and R₂ are S; Q is O; M is O; T is O; n is 1; and A is OH. In yet further embodiment, both R₁ and R₂ are S; Q is O; M is O; T is O; and n is 0. In another embodiment, both R₁ and R₂ are S; Q is O; M is CCl₂; T is O; and n is 1; and A is O.

In one embodiment provided a pharmaceutical composition comprising a Compound having the structure:

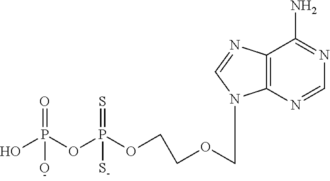

or a pharmaceutically acceptable salt, and at least one pharmaceutically acceptable carrier. In another embodiment, provided a pharmaceutical composition comprising a Compound having the structure:

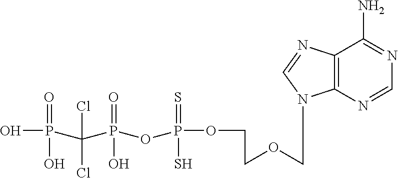 [SMILES: Nc1ncnc2c1ncn2COCCOP(=S)(S)OP(=O)(O)C(Cl)(Cl)P(=O)(O)O]

or a pharmaceutically acceptable salt, and at least one pharmaceutically acceptable carrier. In yet further embodiment, provided a pharmaceutical composition comprising a Compound having the structure:

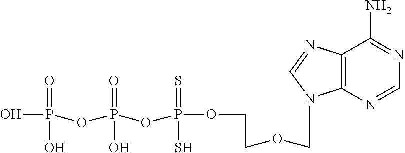 [SMILES: Nc1ncnc2c1ncn2COCCOP(=S)(S)OP(=O)(O)OP(=O)(O)O]

or a pharmaceutically acceptable salt, and at least one pharmaceutically acceptable carrier. In yet further embodiment, provided a pharmaceutical composition comprising a Compound having the structure:

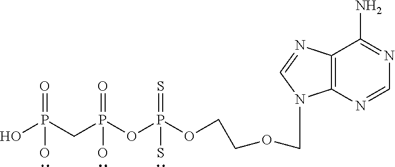 [SMILES: Nc1ncnc2c1ncn2COCCOP(=S)(S)OP(=O)(O)CP(=O)(O)O]

or a pharmaceutically acceptable salt, and at least one pharmaceutically acceptable carrier.

In one embodiment, provided a method for treating a condition associated with enhanced NPP1 activity in a subject in need of such treatment comprising administering to the subject a therapeutically effective amount of the pharmaceutical composition comprising a Compound having the structure,

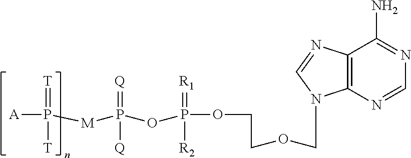

or a pharmaceutically acceptable salt thereof, wherein:

R₁ and R₂ is each independently selected from O and S; Q is selected from O and S; M is selected from O, OH, CH₂, CCl₂, CBr₂, and CF₂; T is selected from O and S; A is selected from O—, OH and a nucleoside; and, n is 0 or 1. In one embodiment both R₁ and R₂ are S; Q is O; M is CH₂; T is O; n is 1; and A is O. In yet another embodiment, both R₁ and R₂ are S; Q is O; M is O; T is O; n is 1; and A is OH. In one embodiment, both R₁ and R₂ are S; Q is O; M is O; T is O; and n is 0. In yet another embodiment, both R₁ and R₂ are S; Q is O; M is CCl₂; T is O; and n is 1; and A is O. In another embodiment of the invention, the condition associated with elevated levels of NPP1 is calcific aortic valve disease (CAVD). In further embodiment, the condition associated with elevated levels of NPP1 is Calcium Pyrophosphate Dihydrate (CPPD) disease.

In one embodiment, provided a method for treating a condition associated with elevated NPP1 levels in a subject in need of such treatment, comprising administering to the subject a therapeutically effective amount of the Compound having the structure,

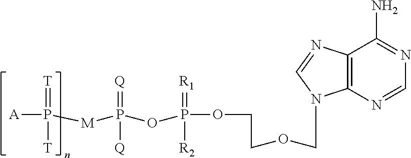

or a pharmaceutically acceptable salt thereof, wherein:

R₁ and R₂ is each independently selected from O and S; Q is selected from O and S; M is selected from O, OH, CH₂, CCl₂, CBr₂, and CF₂; T is selected from O and S; A is O—, OH and a nucleoside; and n is 0 or 1. In one embodiment both R₁ and R₂ are S; Q is O; M is CH₂; T is O; n is 1; and A is O. In yet another embodiment, both R₁ and R₂ are S; Q is O; M is O; T is O; n is 1; and A is OH. In one embodiment, both R₁ and R₂ are S; Q is O; M is O; T is O; and n is 0. In yet another embodiment, both R₁ and R₂ are S; Q is O; M is CCl₂; T is O; and n is 1; and A is O. In another embodiment of the invention, the condition associated with elevated levels of NPP1 is calcific aortic valve disease (CAVD). In another embodiment, the condition associated with elevated levels of NPP1 is Calcium Pyrophosphate Dihydrate (CPPD) disease.

According to one embodiment, provided a compound having the structure,

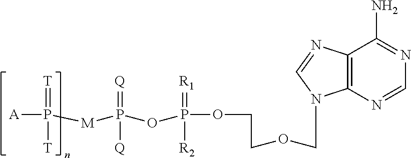

or a pharmaceutically acceptable salt thereof, wherein:

R₁ and R₂ is each independently selected from O and S; Q is selected from O and S; M is selected from O, OH, CH₂, CCl₂, CBr₂, and CF₂; T is selected from O and S; A is selected from O—, OH and a nucleoside; and n is 0 or 1 for use as a medicament. In one embodiment both R₁ and R₂ are S; Q is O; M is CH₂; T is O; n is 1; and A is O. In yet another embodiment, both R₁ and R₂ are S; Q is O; M is O; T is O; n is 1; and A is OH. In one embodiment, both R₁ and R₂ are S; Q is O; M is O; T is O; and n is 0. In yet another embodiment, both R₁ and R₂ are S; Q is O; M is CCl₂; T is O; and n is 1; and A is O. According to some embodiments, provided a pharmaceutical composition of the invention for use in the treatment of a condition associated with enhanced NPP1 activity. According to some embodiments, provided a pharmaceutical composition of the invention for use in the treatment of a condition associated with elevated NPP1 levels.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention.

Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

The term “Alkyl” refers, but not limited to linear or branched alkyl groups having from 1 to 16 carbon atoms. This term is exemplified by groups such as methyl, t-butyl, n-heptyl, octyl and the like. The term “Alkenyl” refers, but not limited to alkenyl group preferably having from 2 to 10 carbon atoms and more preferably 2 to 6 carbon atoms and having at least 1 and preferably from 1-2 sites of alkenyl unsaturation.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms.

A “therapeutically-effective amount” means the amount of a compound that, when administered to a subject for treating a disease, is sufficient to effect such treatment for the disease. The “therapeutically-effective amount” will vary depending on the compound, the disease, and its severity and the age, weight, etc., of the subject to be treated.

As used herein the term “Pharmaceutically-acceptable salt” refers to salts which retain the biological effectiveness and properties of compounds which are not biologically or otherwise undesirable. Pharmaceutically acceptable salts refer to pharmaceutically acceptable salts of the compounds, which salts are derived from a variety of organic and inorganic counter ions well known in the art.

The pharmaceutical dosage forms may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration the invention provides suppositories with hydrophilic or hydrophobic vehicles; for topical application as ointments; and for transdermal delivery the invention provides suitable delivery systems as known in the art.

Tablets may contain suitable binders, lubricants, disintegrating agents, coloring agents, flavoring agents, flow-inducing agents, melting agents, stabilizing agents, solubilizing agents, antioxidants, buffering agent, chelating agents, fillers and plasticizers. For instance, for oral administration in the dosage unit form of a tablet or capsule, the active drug component can be combined with an oral, non-toxic, pharmaceutically acceptable, inert carrier such as gelatin, agar, starch, methyl cellulose, dicalcium phosphate, calcium sulfate, mannitol, sorbitol, microcrystalline cellulose and the like. Suitable binders include starch, gelatin, natural sugars such as corn starch, natural and synthetic gums such as acacia, tragacanth, or sodium alginate, povidone, carboxymethylcellulose, polyethylene glycol, waxes, and the like. Antioxidants include ascorbic acid, fumaric acid, citric acid, malic acid, gallic acid and its salts and esters, butylated hydroxyanisole, editic acid. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

In the examples below, if an abbreviation is not defined above, it has its generally accepted meaning. Further, all temperatures are in degrees Celsius (unless otherwise indicated). The following methods were used to prepare the compounds set forth below as indicated.

### EXAMPLES

### Example 1: Synthesis of uridine 5′-P_{α,α}-dithiophosphate analogues

Uridine-2′,3′-O-methoxymethylidene, 14, was treated with 1,3,2-dithiaphospholane, 15, at −38° C. to obtain intermediate 16. The latter was oxidized by powdered elemental sulfur at RT to give product 13 at 55% yield. Reagents and conditions: (a) DIPEA, CH₃CN, −38° C., 3 h; (b) S₈, overnight, RT.

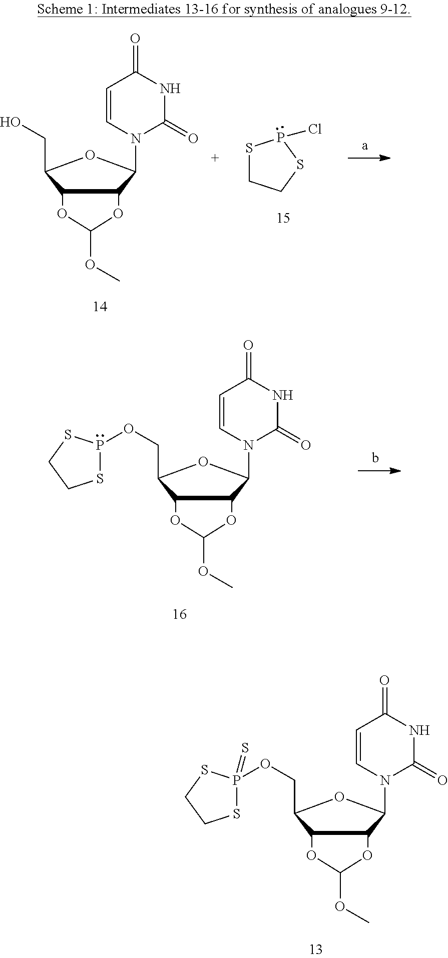

Uridine-P_{α,α}-dithiophosphate analogues 9-12 were synthesized from thio-dithiaphospholane intermediate, 13, via DBU-mediated nucleophilic dithiaphospholane ring-opening. Specifically, orthophosphate/pyrophosphate/methylenediphosphonate was applied as nucleophiles. The reactions were performed at RT with complete exclusion of moisture. was in the range of 0.5-15 h (the longest reaction time was for orthophosphate and the shortest reaction time—for methylenediphosphonate). The nucleophilic ring-opening reaction was followed by deprotection of the methoxymethylidene group. The crude material was separated on an anion exchanger and eluted with a linear gradient of ammonium bicarbonate solution with no need for further purification by HPLC (94-98% purity).

Compounds 10 and 12 were obtained by reaction of 13 with pyrophosphate tris-tributylammonium salt (1 and 0.5 eq. respectively) at 40% and 31% overall yield, respectively. Compounds 9 and 11 were obtained by reaction of 13 with orthophosphate bis-tributylammonium salt or methylenediphosphonate tris-tributylammonium salt at 18% and 44% overall yield, respectively. Reagents and conditions: DBU, CH₃CN, RT, 0.5-15 h

[symbol]

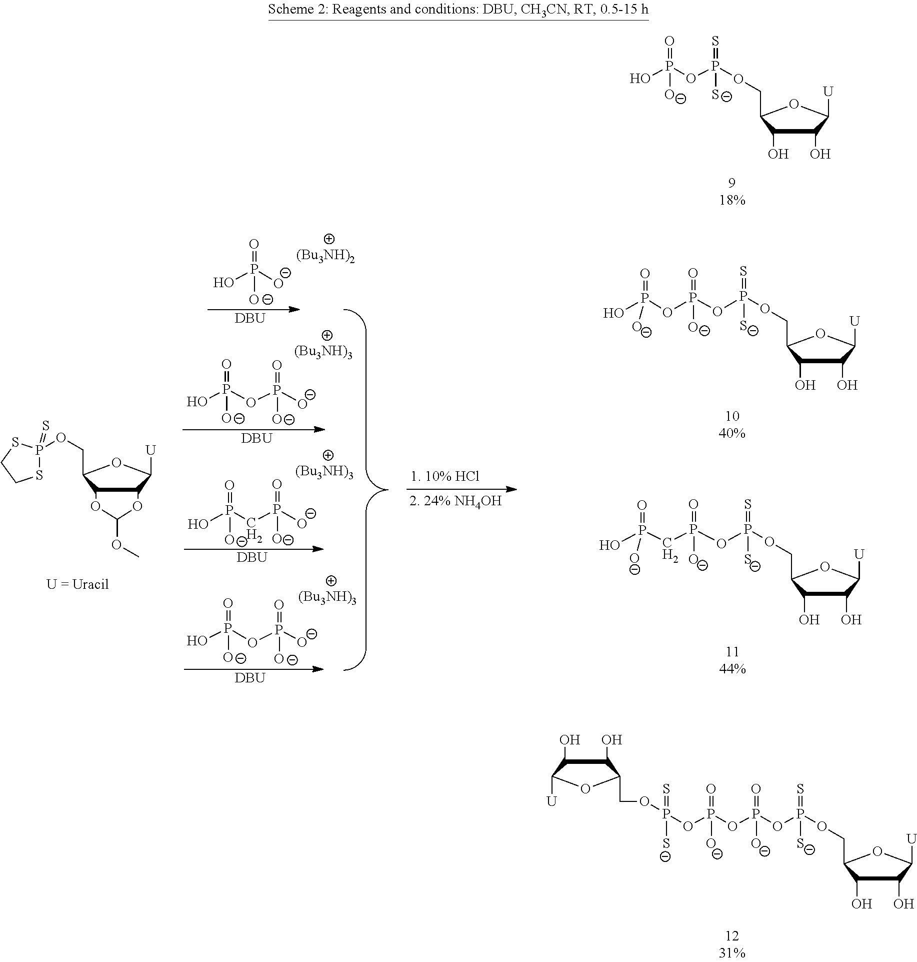

### Example 2: NPP1 Inhibitory Activity of Analogues 9-12

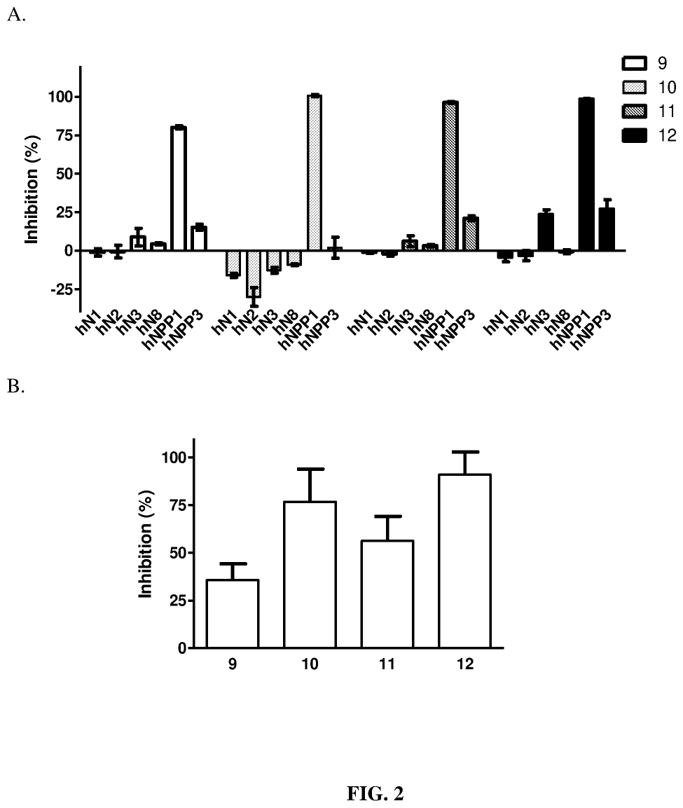

Analogues 9-12 were evaluated for their ability to inhibit human NPP1. We found that all these analogues at 100 μM potently inhibited human NPP1 (80-100% inhibition) using thymidine 5′-monophosphate p-nitrophenyl ester, pNP-TMP, at 100 μM as the substrate (FIG. 2A). In particular, uridine 5′-P_{α,α}—S, S-triphosphate, 10, proved to be a highly potent and selective NPP1 inhibitor (100% inhibition at 100 μM), with no inhibition of the related NPP3 and other ectonucleotidases (NTPDase1, -2, -3, -8).

Analogues 9 and 11 had negligible effects on the ATPase activity of human NTPDases and a minor effect on human NPP3: 15 and 21% inhibition for analogues 9 and 11, respectively. Analogue 12 effectively blocked human NPP1 activity using either pNP-TMP or ATP (100 μM) as the substrates (FIGS. 2A and 2B). Analogue 12 exhibited Ki 27 nM with pNP-TMP as the substrate, being ca. 10- and 40-time more effective than analogues 10 and 11, respectively (Table 1). Analogue 12 was selective to NPP1 vs. human NTPDase1,2,8, but mildly inhibited human NTPDase3 and human NPP3 by 24 and 27%, respectively.

Results:

At least 2 independent experiments were performed. In panel A, the activity with 100 μM ATP was set as 100%, which was 287±31, 395±67, 278±22, 119±26 nmol of Pi·min₋₁·mg of protein₋₁ for human NTPDase1, -2, -3 and -8, respectively, and the activity with 100 μM pNP-TMP was set as 100% which was 67±5, 21±1 nmol of p-nitrophenol·min₋₁·mg of protein₋₁ for human NPP1 and NPP3, respectively. In panel B, the activity with 100 μM ATP was set as 100% which was 34±7 nmol of PPi·min₋₁·mg of protein₋₁ for NPP1.

| Inhibitor | IC₅₀ (μM) | Ki (μM) |
|---|---|---|
| 10 | 1.2 ± 0.1 | 0.24 ± 0.02 |
| 11 | 4.3 ± 0.1 | 1.11 ± 0.19 |
| 12 | 0.125 ± 0.004 | 0.027 ± 0.009 |

Table 1. IC₅₀ and Ki values obtained for NPP1 inhibition by the promising analogues 10-12 using pNP-TMP as a substrate.

Data presented are the mean±SEM of at least 3 independent experiments.

### Example 3: Stability of Analogues 9-12 to Degradation by NPP1 and NTPDases

Uridine 5′-P_{α,α}-phosphorodithioate analogues, 9-12, were tested for their stability to degradation by NPP1 and NTPDases. These enzymes are involved in the metabolism of extracellular nucleotides. Analogues 9-12 were not hydrolyzed by hNPP1 (Table 2). Analogues 9, 11, and 12 resisted hydrolysis by hNTPDase1, -2, -3, -8 (Table 2). Analogue 10 was hydrolyzed by hNTPDase1, -2 and -3 at 30-39% the rate of ATP and was slightly hydrolyzed by human NTPDase8 at 16% the rate of ATP.

TABLE 2
Stability of analogues 9-12 to hydrolysis by human ectonucleotidases

| | Relative activity (%) ± SEM of ATP hydrolysis | | | |
|---|---|---|---|---|
| ectoenzymes | 9 | 10 | 11 | 12 |
| NTPDase1 | 0 | 30.8 ± 3.2 | 0 | 2.7 ± 0.4 |
| NTPDase2 | 0.6 ± 0.2 | 32.0 ± 3.3 | 0 | 2.0 ± 0.2 |
| NTPDase3 | 1.0 ± 0.2 | 39.7 ± 1.9 | 0 | 3.2 ± 0.6 |
| NTPDase8 | 0.2 ± 0.3 | 16.5 ± 2.8 | 0 | 1.1 ± 0.6 |
| NPP1 | 0 | 0 | 0 | 0 |

Analogues 9-12 were incubated in the presence of the indicated ectonucleotidase at a concentration of 100 μM. The activity with 100 μM ATP was set as 100%, which was 329±108, 302±34, 263±51, 195±76 nmol of Pi·min₋₁·mg of protein₋₁ for NTPDase1, -2, -3 and -8, respectively, and 34±7 nmol for NPP1. Data presented are the mean±SEM of results from two experiments.

### Example 4: Effects of Uridine-5′-P_{α,α}-Dithiophosphate Analogues on the Uracil Nucleotide Responding P2Y_{2,4,6} Receptors

Uridine nucleotides are not only substrates of ecto-nucleotidases and are also agonists of P2Y_{2,4,6}-Rs, and thus could potentially exert additional biological effects. For instance, activation of P2Y₂ receptor results in the inhibition of bone formation.

To evaluate NPP1 selectivity of analogues 9-12, we tested them also for their ability to activate the uridine-nucleotide sensitive human recombinant P2Y_{2,4,6}-receptors stably expressed in AD293 cells. The compounds were tested at 100 μM and P2YR activation was determined by measuring variations in intracellular calcium concentration (Δ[Ca₂₊]ᵢ) using UDP or UTP as control as described.

Results:

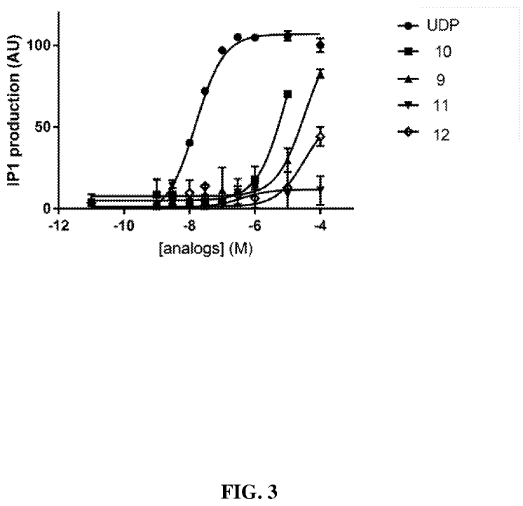

All of the compounds had no effect at P2Y₂ and P2Y₄-Rs, and a weak agonist activity at P2Y₆—R. Then, we switched to a more sensitive assay that measures the accumulation of inositol-1-phosphate (IP1), which is also directly linked to G_{q} activation.₃₂ Analogues 9, 10 and 12 showed limited agonist activities at P2Y₆—R, whereas 11 was devoid of any P2Y₆—R activity (FIG. 3).

Analogues 9, 10, 11 and 12 did not have any, or very weak, agonistic activities at uracil nucleotide responding receptors when compared to their natural ligands. Unlike the previously reported uridine-5′-P_{α}—S-triphosphate diastereomers, which are highly potent agonists at P2Y₂—R and P2Y₄—R,₂₆ the presence of two sulfur atoms at P_{α}-phosphate, as in compounds 9-12, resulted in loss of activity at both P2Y₂—R and P2Y₄—R.

### Example 5: Docking Simulations of Analogues 9-12 at Human NPP1 and NPP3 Molecular Models

Docking simulations were used to provide insight into the inhibitory activity and NPP1 selectivity of the analogues studied here. Homology models of human NPP1 and NPP3 based on the crystal structures of mouse NPP1 were used.₃₃

First, the ability of Glide to reproduce the binding mode of AMP in the crystal structure of mouse NPP1 (PDB code 4GTW) was confirmed. This binding mode was indeed reproduced to within 1.1 Å based on the pose with the most favorable (lowest) emodel energy (emodel is the recommended scoring function for comparing different poses of the same ligand; see https://www.schrodinger.com/kb/1027 suggesting that Glide is a suitable docking tool for this system.

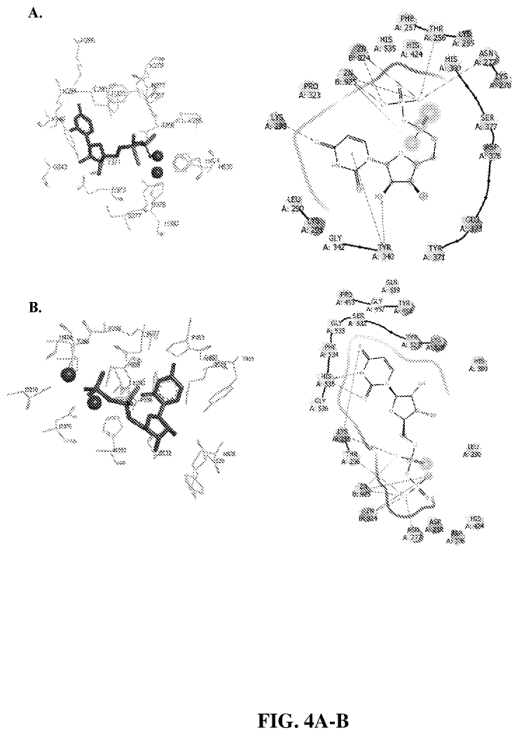

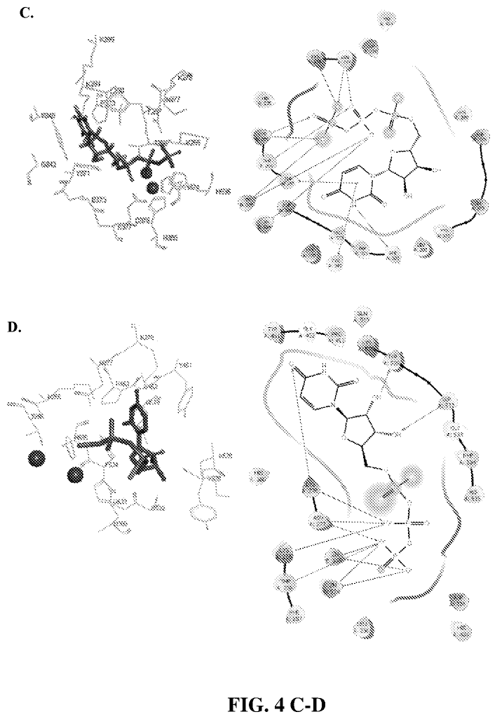

Next, analogues 9-12 were docked into the hNPP1 and hNPP3 binding-sites and their lowest energy poses were analyzed. The results are presented in FIG. 4, and Table 3.

Results:

In general, two types of binding modes were observed: one that highly resembles the binding mode of AMP in the crystal structure of mouse NPP1 (AMP-like conformations), and binding mode where the tri-phosphate moiety maintains its chelation to zinc-ion, but the nucleotide moiety adopts an alternative conformation. In the case of NPP1, all ligands adopt both AMP-like and non-AMP-like conformations (FIG. 4).

These binding modes suggest that all analogues could compete with ATP for binding-site interactions at NPP1. Analogues 10 and 12 demonstrated preference to chelation of zinc-ions via their phosphate non-bridging oxygen atoms for both NPP1 and NPP3, whereas analogues 9 and 11 demonstrated no preference for S-chelation over O-chelation for NPP1 and preference for O-chelation for NPP3. Other poses for all analogues feature either O- or S-chelation for the two proteins.

Glide predicted analogue 12 to have the highest affinity to NPP1, followed by analogues 9-11 which all have similar Glide scores (Table 3). The improved binding of analogue 12 to the NPP1 binding-site could be attributed to enhanced electrostatic interactions (primarily salt bridges and H-bonds, FIG. 4G). Thus analogues 9-12, participate in interactions with binding site residues as follows: 9 one electrostatic interaction (Lys255) and five hydrogen bonds (Lys255, Thr256, Asn277, and His535); 10: two electrostatic interactions (Lys255, Lys278) and five hydrogen bonds (Lys255, Thr256, Asn277, and Phe321); 11: two electrostatic interactions (Lys255) and four hydrogen bonds (Asp218, Lys255, Asn277, and Ser377); 12: two electrostatic interactions (Lys255, Lys278) and six hydrogen bonds (Thr256, Asn277, Leu290, Lys291, and Tyr371) (see Table 3).

Furthermore, all analogues were predicted to be poorer binders to NPP3 vs. NPP1 (Table 1 and FIG. 2). A comparative examination of the interaction patterns (FIG. 4 and Table 3) suggests that similar to the trend observed in NPP1 potency, NPP1 selectivity could be primarily attributed to the effect of electrostatic interactions. Thus, all analogues form a larger number of salt bridges and H-bonds with the NPP1 site than with the NPP3 site.

The ligand binding site in NPP1 is enriched with Lys residues, unlike the ligand binding site in NPP3 (7 vs. 1 Lys residues in NPP1 and NPP3, respectively). The electrostatic potential in the ligand binding site of NPP1 is therefore more positive than in that of NPP3, making it a more suitable binding-site for the negatively charged nucleotides.

Analogues 9-12 presented both AMP-like and non-AMP-like binding modes at NPP1 and NPP3. The di-uridine-tetrathio-tetraphosphate analogue 12 was shown to occupy both binding-regions simultaneously. The resulting multiple binding interactions (Table 3), possibly contribute to the enhanced potency and selectivity of this analogue.

TABLE 3
Docking scores and list of interactions of AMP-like and non-AMP-like binding modes of analogues
9-12 at the human NPP1 and NPP3 homology models.

| | | gScore NPP1 | List of | gScore NPP3 | List of |
|---|---|---|---|---|---|
| Comp | Binding Mode | (kcal/mol) | Interactions | (kcal/mol) | Interactions |
| 9 | AMP-like | −9.3 | □□□ | −7.6 | □□□ |
| | | | interactions: Y340 | | interactions: F206, Y289 |
| | | | H bonds: T256, | | H bonds: T205, N226 |
| | | | N277, K295 | | |
| | Non AMP-like | −9.9 | H bonds: K255, | — | — |
| | | | T256, N277, H535 | | |
| | | | Electrostatic: K255 | | |
| 10 | AMP-like | −10.1 | □□□ | −7.9 | □□□ |
| | | | interactions: | | interactions: Y289 |
| | | | F257, Y340 | | H bonds: T205, N226 |
| | | | H bonds: K255, | | Electrostatic: K204 |
| | | | T256, N277, F321 | | |
| | | | Electrostatic: | | |
| | | | K255, K278 | | |
| | Non AMP-like | −9.5 | H bonds: T256, | — | — |
| | | | N277, K278, | | |
| | | | Y529, S532 | | |
| | | | Electrostatic: | | |
| | | | K255, K278 | | |
| 11 | AMP-like | −9.9 | H bonds: D218, | −8.6 | □□□ |
| | | | N277, S377, H535 | | interactions: F206 |
| | | | Electrostatic: K255 | | H bonds: N226, Q244, |
| | | | | | G273, E275 |
| | Non AMP-like | −9.8 | H bonds: T256, | −5.2 | H bonds: T205, N226, |
| | | | N277, K278, | | Y289, S326, T476 |
| | | | Y451, Q519, | | |
| | | | Y529, S532, | | |
| | | | H535 | | |
| 12 | Dinucleotide | −11.7 | □□□ | −10.9 | □□□ |
| | | | interactions: F257, | | interactions: Y289 |
| | | | Y340 | | H bonds: H329, N477 |
| | | | H bonds: T256, | | Electrostatic: K204 |
| | | | N277, L290, | | |
| | | | K291, Y371 | | |
| | | | Electrostatic: K255, | | |
| | | | K278 | | |

### Example 6: Evaluation of the Chemical Stability of Uridine-5′-P_{α,α}-Dithiophosphate Analogues

To evaluate the chemical stability of uridine-5′-P_{α,α}-dithiophosphate analogues, we measured by ₃₁P-NMR the time-dependent percentage of the analogues remaining under basic and acidic conditions, and due to air-oxidation.

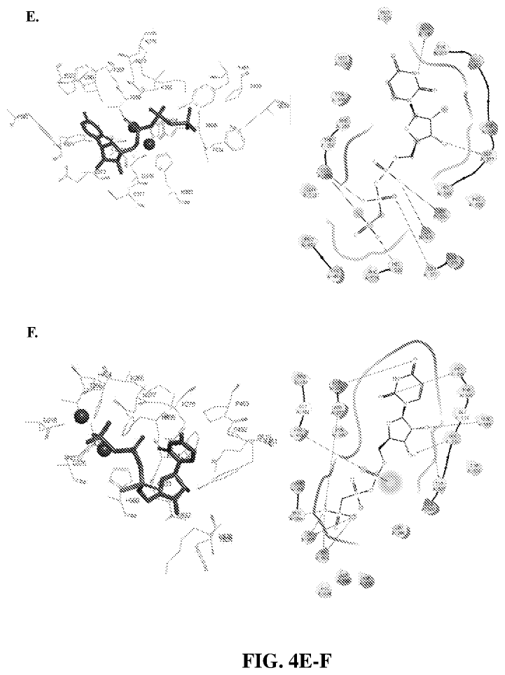

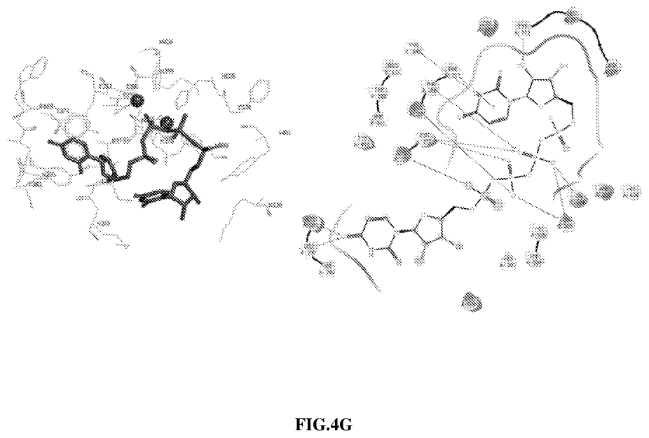

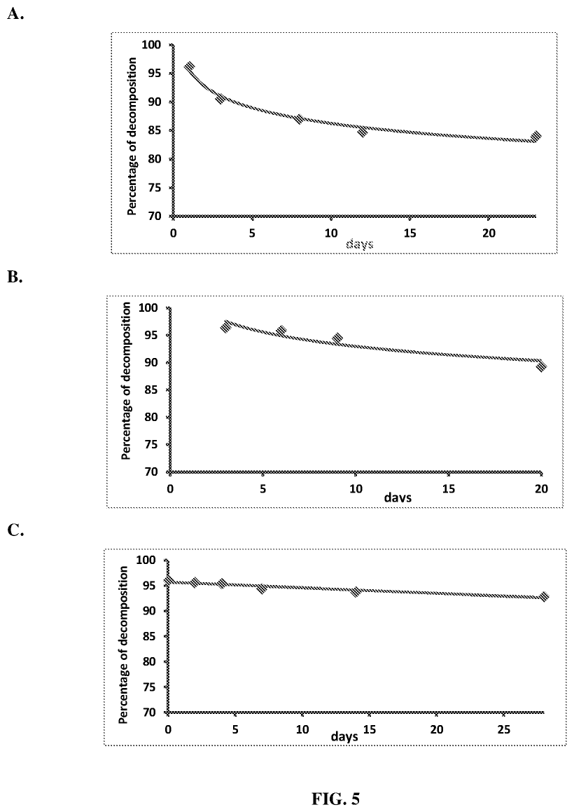

Specifically, the stability of UTP-5′-P_{α,α}-dithiophosphate, 10, at RT and pD 2.1 (pH 1.7) was monitored for 23 days. During that time only 15% of the starting material decomposed (FIG. 5A). Compound 10 under these acidic conditions was slightly less stable than the parent compound, UTP, 11% of which decomposed after 20 days (FIG. 5B), indicating that the phosphorodithioate moiety does not significantly reduce the chemical stability of UTP. At pD 2.1 UDP was more stable than UTP (only 7% decomposition after 28 days) (FIG. 5C).

Mass spectrum (ESI negative) and ₃₁P-NMR analysis of freeze-dried 10 after 23 days at pD 2.1, revealed hydrolysis products, apart from 10, uridine-5′-P_{α,α}-dithiodiphosphate, 9, and inorganic phosphate.

Results:

Orally administered pharmacologically active molecules should resist acidic and slightly basic hydrolysis in the different parts of the gastro-intestinal tract and should be stable against oxidizing agents. The newly synthesized uridine-5′-P_{α,α}-dithiophosphate analogues have a number of chemical functionalities that could be hydrolyzed under acidic or basic conditions including phosphodiester bonds, glycosidic bond, and a dithiophosphate moiety that can undergo desulfurization. The latter moiety may also undergo oxidation to the corresponding disulfide product.

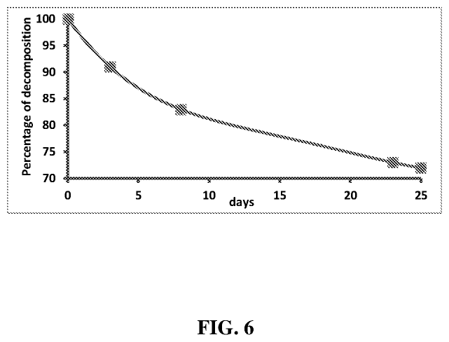

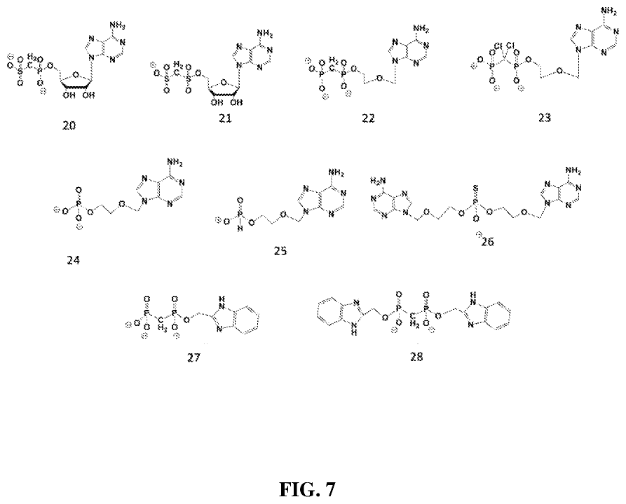

Compound 10 was found to be relatively stable also under basic conditions, pD 12.1. After 25 days, ₃₁P-NMR spectrum showed that only 24% of 10 underwent decomposition (FIG. 6). The hydrolysis products included uridine-5′-P_{α,α}-dithiodiphosphate, 9; uridine-5′-P_{α,α}-dithio-monophosphate, 17; inorganic dithio-phosphate, 18; UMP, 19; and uridine-5′-P_{α,α}-dithio-(mercapto-ethylene)monophosphate, 20, resulting from dithiaphopholane ring opening without thiirane release; and inorganic phosphate. The phosphorodithioate moiety in 17 and 18 remained intact and did not undergo basic hydrolysis to the corresponding phosphorothioate and phosphate analogues. Under these basic conditions 10 is less stable than UTP (which exhibited only 10% decomposition after 20 days).

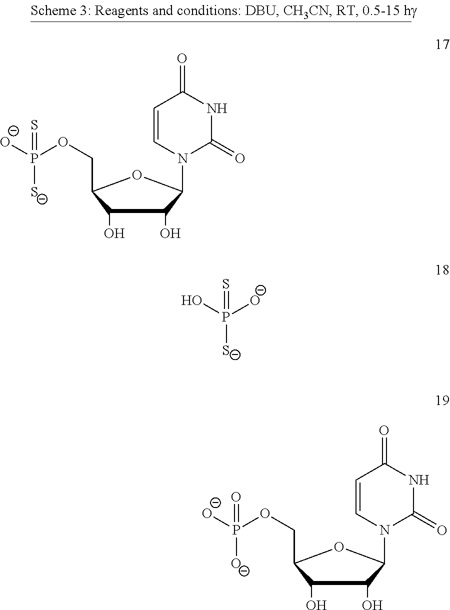

Compound 10 also exhibited remarkably high stability to air-oxidation. An aqueous solution of 10 was subjected to a constant air flow. After three weeks at RT ₃₁P-NMR spectrum of the solution showed no new signals, indicating no tendency to disulfide bond formation due to air-oxidation.

When compound 12 was subjected to pD 1.8 (pH 1.4) for 16 days at RT, ₃₁P-NMR spectrum showed only 6% decomposition of 12. The only hydrolysis product was inorganic phosphate, probably due to a series of hydrolytic steps described in Scheme 4. Compound 12 was found to be relatively stable also under basic conditions, pD 12.6. After 16 days, ₃₁P-NMR spectrum showed a loss of only 13% of 12, and formation of uridine-5′-P_{α,α}-dithio-monophosphate, 17, inorganic dithiophosphate, 18, and inorganic phosphate.

The decomposition products of 12 under basic conditions differed from those under acidic conditions, indicating a much more rapid conversion of dithiophosphate to inorganic phosphate in acidic environment, than under basic conditions. Uridine-5′-P_{α,α}-dithiomonophosphate, 17, and inorganic dithiophosphate, 18, are intermediates giving rise to inorganic phosphate. In addition, compound 12 was completely resistant to air-oxidation for at least 3 weeks.

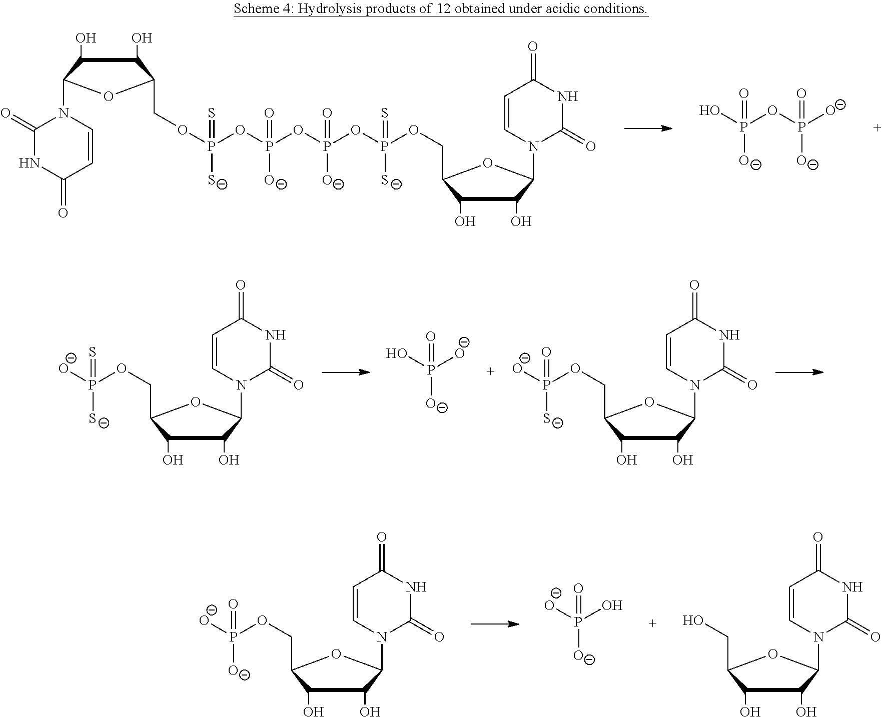

Likewise, 11 showed only 6% decomposition after 25 days at pD 2.1. Hydrolysis products included methylenediphosphonate, 21, and inorganic phosphate. These products are due to hydrolysis of the phosphodiester bond between the diphosphonate moiety and P_{α,α}-dithiophosphate followed by hydrolysis of the dithiophosphate group. The absence of hydrolysis products containing a dithio- or thiophosphate moiety is probably due to further hydrolysis of sulfur-containing intermediates to orthophosphate (Scheme 5).

Compound 11 was found to be relatively stable also under basic conditions, pD 12.1. After 25 days at RT, only 23% of 11 underwent decomposition giving rise to uridine-5′-P_{α,α}-dithiomonophosphate, 17, inorganic dithio-phosphate, 18, uridine-5′-P_{α,α}-dithio-(mercapto-ethylene)-monophosphate, 20; and diphosphonate 21. Under neutral pH (air-flow, 3 weeks) 11 showed complete stability.

In summation, uridine-5′-Pα,α-dithiophosphate analogues demonstrated a much greater stability under acidic and basic conditions than the related nucleotides, 2-benzylthio-ATP-P_{α}—S and 2-methylthio-ADP-P_{α}—S._{24,34} This increased stability likely results from the additional sulfur atom attached to the Pa, possibly, due to the greater volume of the dithiophosphate vs. thiophosphate moiety which sterically hinders attack by a water molecule or hydroxide ion.

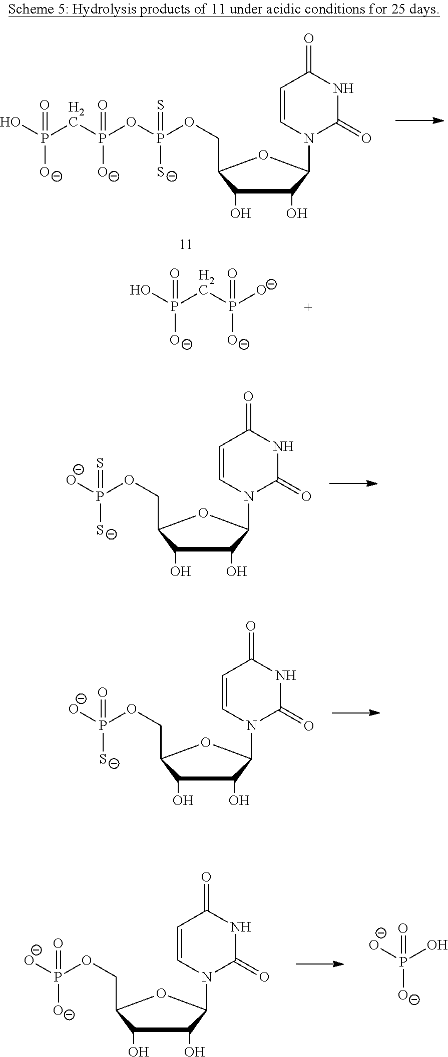

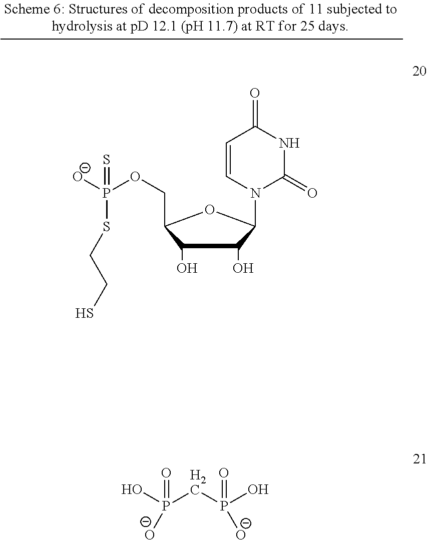

As expected, the most stable analogue of this series under acidic conditions was 11, due to replacement of P_{β},P_{γ}-bridging oxygen atom by a methylene group (Table 4). Under basic conditions, di-uridine-(5′, 5″-P_{α,α}-dithio)-tetraphosphate, 12, showed the highest stability, due to the absence of a terminal phosphate group. Under oxidizing conditions compounds 10-12 were completely stable for at least 3 weeks. This increased stability to oxidizing conditions possibly resulted from steric hindrance at P_{α} phosphate.

Apparently, the phosphorodithioate moiety in analogue 10, only slightly reduced the chemical stability as compared to UTP, unlike the related 5′-P_{α}-thiophosphate-containing compounds where the thiophosphate group significantly lowered the stability compared to the parent compounds (Table 4)._{24,34}_ENREF_1_ENREF_4 Yet, compounds 11 and 12 showed higher stability than that of UTP.

TABLE 4
Time required for decomposition of 5% and 10% of all evaluated compounds.

| | | Basic pH | | | | Acidic pH | | | |
|---|---|---|---|---|---|---|---|---|---|
| | | Time for | | Time for | | Time for | | Time for | |
| | | decomposition | | decomposition | | decomposition | | decomposition | |
| No. | Compound | of 5% | | of 10% | | of 5% | | of 10% | |
| 1 | UTP | 1 | d | 8.5 | d | 7.5 | d | 19 | d |
| 2 | 10 | 2 | d | 3 | d | 1.5 | d | 3 | d |
| 3 | 11 | 2.5 | d | 5.5 | d | 15 | d | >25 | d |
| 4 | 12 | 4.5 | d | 10 | d | 1 | d | >16 | d |

| | | | | | | | |
|---|---|---|---|---|---|---|---|
| 5 | 2-Benzylthio- | N/A | N/A | 0.8 | d | 1.7 | d |

| | ATP-α-S₃₄ | | | | | | |
|---|---|---|---|---|---|---|---|
| 6 | 2-Methylthio- | <0.5 | h | <0.5 | h | N/A | N/A |

ADP-α-S₂₄

Discussion:

Calcific aortic valve disease and calcium pyrophosphate dihydrate disease, whose pathology is related to abnormally high level of NPP1, are considered as unmet medical needs. Therefore, the need for biocompatible, potent, selective, and stable NPP1 inhibitors is urgent. Here, we developed novel NPP1 inhibitors based on uridine 5′-P_{α,α}-di-thio-phosphate analogues, 9-12. These analogues were designed to maintain the desired pharmacological properties of adenosine 5′-P_{α}-phosphorothioate analogues, which were reported before while avoiding the presence of a chiral center at P_{α}-phosphate group, and the waste of half of the nucleotide product. Analogues 9-12 proved to be highly stable to air-oxidation and significantly chemically stable to acidic and basic pH, turning them into good candidates for further drug development. These findings prompted the evaluation of the inhibitory activity of analogues 9-12 at NPP1. All these analogues potently inhibited hNPP1 (80-100% inhibition) at 100 μM, with nearly no effect on the related NPP3, other ectonucleotidases (NTPDase1,2,3,8), or uridine nucleotide sensitive purinergic receptors (P2Y_{2,4,6}-Rs). The most promising analogue was diuridine-5′-P_{α,α}, 5″-P_{α,α}-tetrathio-tetraphosphate, 12, exhibiting Ki of 27 nM. Docking simulations have indicated that the enhanced NPP1 inhibitory activity and selectivity of 12 could be due to more interactions formed between analogue 12 and the NPP1 site than between any of the other analogues and the NPP1 site. Likewise, more interactions are formed between analogue 12 and the NPP1 vs. the NPP3 site. In summary, analogue 12 represents a highly active, selective, and stable NPP1 inhibitor.

### Example 7: Synthesis of adenosine-5′-(phosphoryl)methylene-sulfonate, 20, and adenosine-5′-(sulfonyl)methylene-sulfonate, 21

Reagents and conditions: (a) 1) Chloromethylene phosphorus dichloride, dry pyridine, Et3N, 0° C., 5 h, 2) 0.2 M triethylammonium bicarbonate (TEAB), pH 8, 2 h, RT (b) Na₂SO₃, microwave, 120° C., 4.5 h (c) 1) 10% HCl (pH 2.3), RT, 3 h; 2) 24% NH4OH, RT, 45 min; 3) Dowex 50WX8 hydrogen form, 10% NaOH. (d) 1) Methanedisulfonyl dichloride, dry pyridine, dry dichloromethane (DCM), 0° C., 4 h 2) 0.2 M TEAB, 2 h, RT.

Adenine-N9-(methoxy)ethyl-BR-bisphosphonate, 22 was targeted as a flexible analog of adenosine 5′-diphosphonate. Methylenediphosphoryl-dichloride was used to introduce a methylene bis-phosphonate moiety at the primary alcohol of the acyclic adenine 32. Product 22 was obtained in 22% yield upon hydrolysis with triethylammonium bicarbonate (TEAB) solution (Scheme 7).

Adenine-N9-(methoxy)ethyl-β ((dichloromethylene)bisphosphonate), 23, was synthesized as follows: adenine-(methoxy)ethanol, 32, was treated with 4-toluene-sulfonyl chloride to give 22, followed by displacement of the tosylate group with di-chloro-methylene bis-phosphonate to yield 23 in 25% yield (Scheme 8).

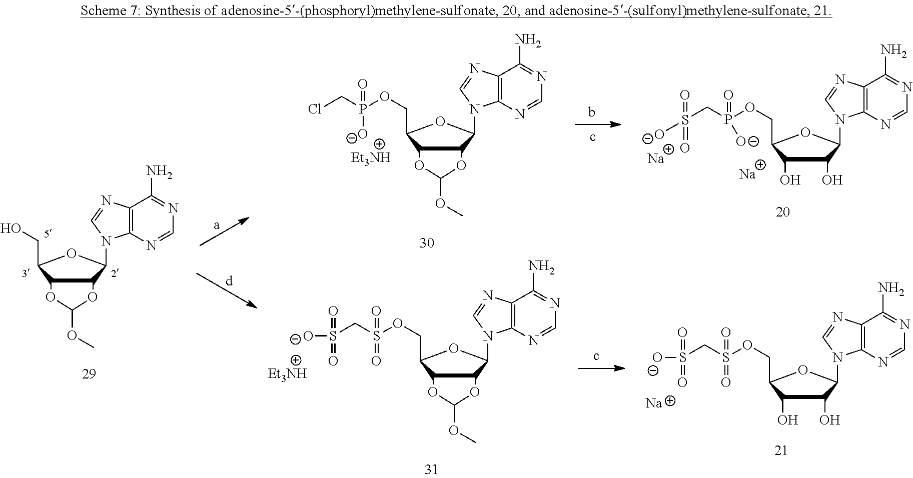

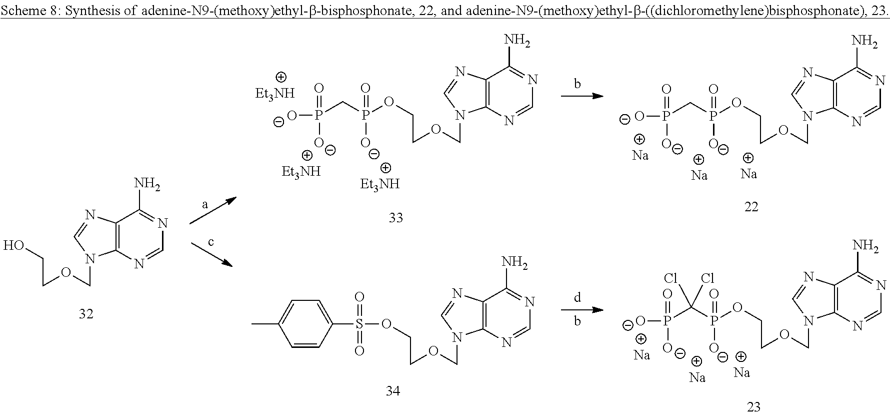

### Example 8: Preparation of adenine-N9-(methoxy)ethyl-β-phosphate, 24

32 was treated with phosphoryl chloride to introduce a monophosphate at the terminal alcohol, followed by hydrolysis with TEAB solution to give 12 in 15% yield (Scheme 9).

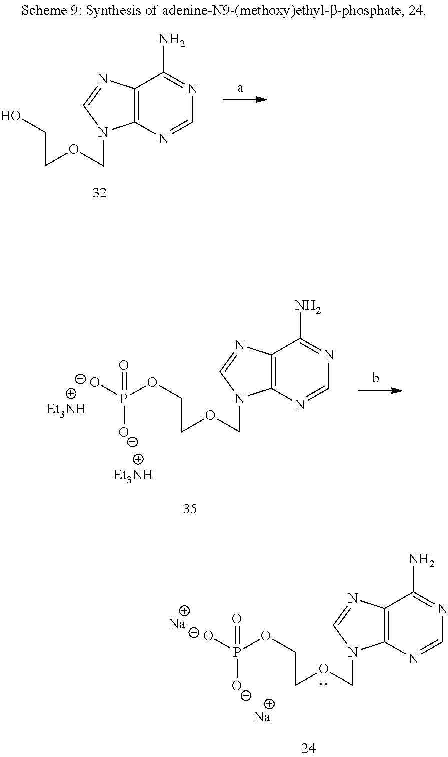

### Example 9: Preparation of adenine-(methoxy)ethyl-β-(H-phosphonate) and Adenine-(methoxy)ethyl-β-(thiophosphate)

For the preparation of adenine-(methoxy)ethyl-β-(H-phosphonate), 25, 32 was treated with 2-chloro-4H-1,3,2-benzo-dioxaphosphorin-4-one followed by hydrolysis with TEAB solution (Scheme 10). Product 25 was obtained in 24% yield. Adenine-(methoxy)ethyl-β-(thiophosphate), 26, was targeted as a flexible analog of adenosine-5′-thio-phosphate, which may in addition improve binding interactions with the NPP1 catalytic zinc ions. We used 2-chloro-4H-1,3,2-benzodioxaphosphorin-4-one to introduce a phosphite group at the terminal hydroxyl residue, followed by oxidation with elemental sulfur and finally hydrolysis with TEAB solution (Scheme 10). Product 26 was obtained in 9% yield.

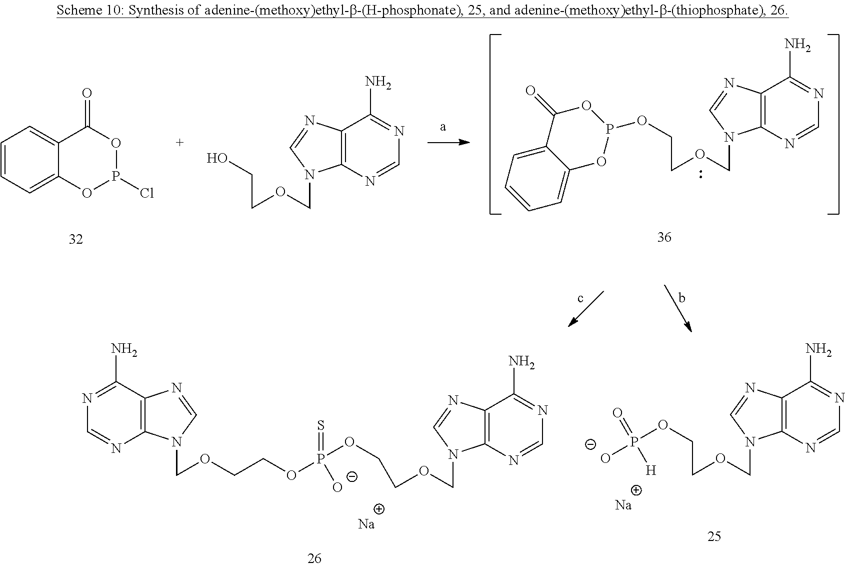

### Example 10: Preparation of Benzimidazol-2-yl-methanol-bisphosphonate

Benzimidazol-2-yl-methanol-bisphosphonate, 27, was prepared as an analog of compound 22. Compound 27 was obtained in 31% yield by treating benzimidazol-2-yl-methanol with methylenediphosphoryl-dichloride, followed by hydrolysis with TEAB solution (Scheme 11). When we treated benzimidazol-2-yl-methanol with 0.5 eq methylene bis(phosphonic-dichloride), we obtained benzimidazole dimer 28 in 8.2% yield.

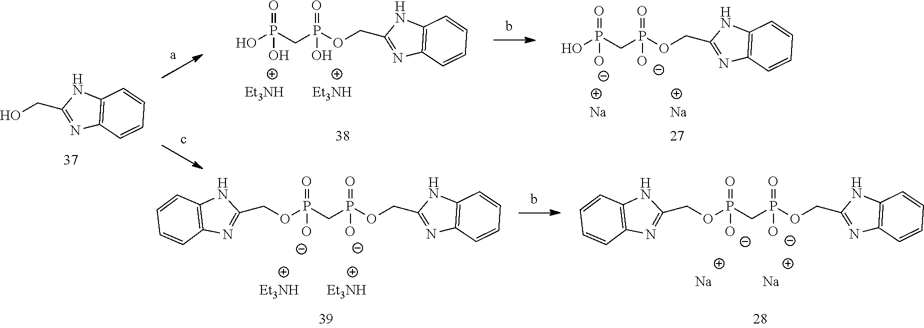

### Example 11: Evaluation of the Ability of Analogs 8-16 to Inhibit Human NPP1 and Other Ectonucleotidases

TABLE 5
Evaluation of inhibitory activities of test compounds at various ectonucleotidases.

| | Ki ± SD (μM) | | | |
|---|---|---|---|---|
| | (% inhibition ± SD at 10 μM) | | | |
| | | | Human CD39 _{b} | |
| Compound | Human NPP1 ₐ | Human NPP3 ₐ | (NTPDase1) | Human CD73 _{c} |
| 20 | inactive_{d} | inactive_{d} | inactive_{d} | 49.5 ± 0.7 |
| 21 | >10 (12 ± 1) | inactive_{d} | inactive_{d} | >10 (23 ± 3) |
| 22 | 16.3 ± 2.5 | inactive_{d} | >10 (22 ± 2) | 12.6 ± 1.4 |
| 23 | >10 (4 ± 1) | inactive_{d} | >10 (24 ± 3) | >10 (17 ± 4) |
| 24 | inactive_{d} | inactive_{d} | inactive_{d} | >10 (5 ± 3) |
| 25 | >10 (6 ± 1) | inactive_{d} | >10 (33 ± 4) | inactive_{d} |
| 26 | >10 (13 ± 1) | inactive_{d} | >10 (15 ± 2) | inactive_{d} |
| 15 | >10 (21 ± 2) | inactive_{d} | inactive_{d} | >10 (18 ± 6) |
| 16 | inactive_{d} | >10 (5 ± 1) | >10 (9 ± 3) | >10 (18 ± 5) |
| AOPCP | 1.28-16.5ₑ | inactive_{d} | >10 (12 ± 5) | 0.197 _{f} |
| Reactive | 0.52 _{g} | 0.71 _{g} | 20.0 ₕ | 3.07 ᵢ |
| Blue 2 | | | | |
| Suramin | 0.26 _{g} | 0.04 _{g} | 300 ₕ | n.d. ⱼ |

ₐ Evaluation of enzyme inhibition using 100 μM p-Nph-5′-TMP as a substrate
_{b} Evaluation of enzyme inhibition using 100 μM ADP as a substrate
_{c} Evaluation of enzyme inhibition using 5 μM [2,8−₃H]AMP as a substrate
_{d}No inhibition at 10 μM
ₑLiterature value [15]
_{f} Literature value [45]
_{g} Literature value [46]
ₕ Literature value [18]
ᵢ Literature value [45]
ⱼ not determined

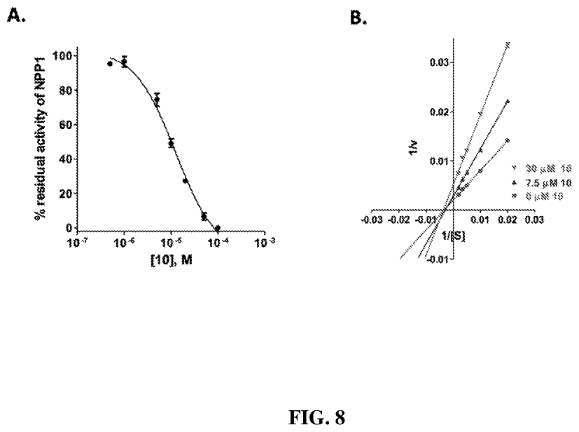

Reference is now made to FIG. 8 demonstrating Concentration-inhibition curve of 22 at human NPP1 vs. 100 μM p-Nph-5′-TMP as a substrate (Ki=16.3±2.5 μM) and Lineweaver-Burk plot of NPP1 inhibition by 22. S, substrate concentration of p-Nph-5′-TMP (μM), v, velocity of enzyme (nmol/min/mg protein) (A); and concentration of 10: green circle, 0 μM; blue triangle, 7.5 μM; violet triangle, 30 μM. Each experiment was performed in triplicates (B).

Reference is now made to FIG. 9 demonstrating Concentration-inhibition curve of 22 at human NPP1 vs. 100 μM ATP as a substrate (Ki=9.60±2.84 μM) and Lineweaver-Burk plot of NPP1 inhibition by 22. S, substrate concentration of ATP (μM), v, velocity of enzyme (nmol/min/mg protein) (A); concentration of 22 green circle, 0 μM; blue triangle, 50 μM; violet triangle, 150 μM (B). Each experiment was performed in triplicates.

### Example 12: Evaluation of the Ability of Analogs 8-16 to Inhibit NPP1 as Compared to TNAP in Human Chondrocytes

It was demonstrated that NPP1 is expressed in primary osteoarthritic human chondrocytes obtained from OA patients undergoing total knee replacement.[36]

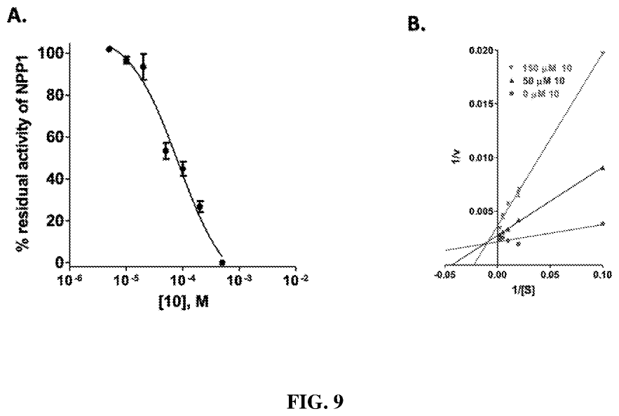

the ability of analogs 20-28 to inhibit NPPase activity in primary human chondrocytes cultured in monolayers at equimolar analog concentrations (100 μM) was evaluated and is demonstrated on FIG. 10A. Out of the studied series of analogs, compound (at 100 μM) displayed the most promising inhibitory capabilities, showing 94% inhibition of NPPase activity in chondrocytes. To confirm that analogs 20-28 do not interfere with tissue non-specific alkaline phosphatase (TNAP) activity in adult human cartilage, their effects on primary human chondrocytes were tested. It was found that analogs 20-28 do not inhibit TNAP activity as measured by p-nitrophenyl phosphate hydrolysis in human chondrocytes as demonstrated on FIG. 10B.

### Example 13: Evaluation of Toxicity of Analogs 8-16 in Primary Human Chondrocytes

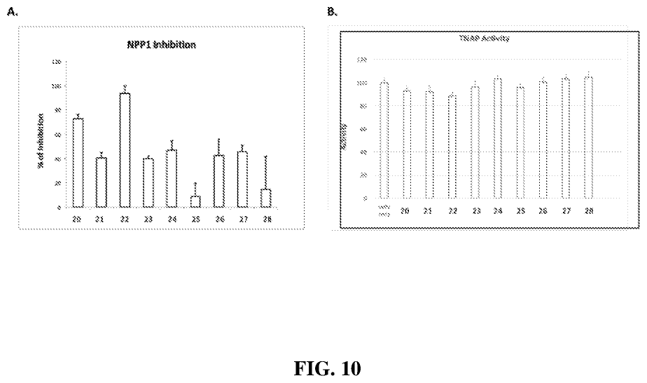

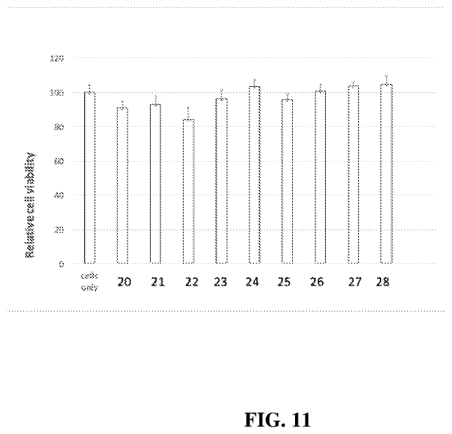

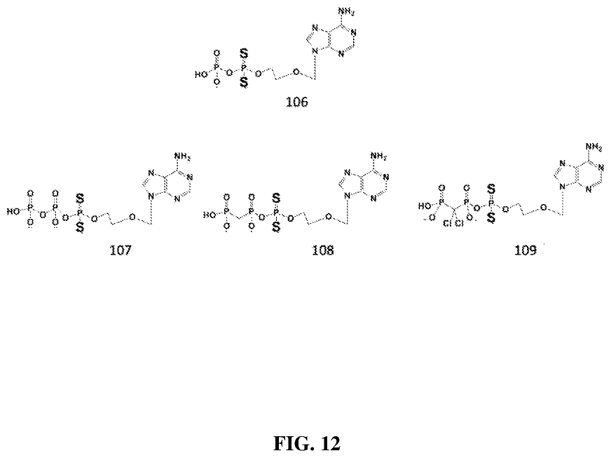

A prerequisite for the application of inhibitors 20-28 as therapeutic agents is the lack of toxicity. For this purpose, primary human chondrocytes were cultured with analogs 20-28 at a high concentration of 1 mM for 24 h. Thereafter, cell viability was measured relative to untreated controls by the XTT assay and is represented on FIG. 11. No significant decrease in cell's viability was discernible at concentrations of up to 1 mM.

### Example 14: Synthesis of Acrylic Adenine-5′-Pα,α-Dithiophosphate Second Generation Analogues

Adenine-(methoxy)-ethoxy-dithiaphospholane, 106 was synthesized by treating adenine-(methoxy) ethanol 110 with chloro1,3,2-dithiaphospholane to form thio-dithiaphospholane intermediate, 111, followed by DBU-assisted nucleophilic ring opening by orthophosphate (Scheme 12). Analog 6 was obtained in 29% yield after elution from an anion exchanger with a linear gradient of ammonium bicarbonate solution, followed by further purification by HPLC.

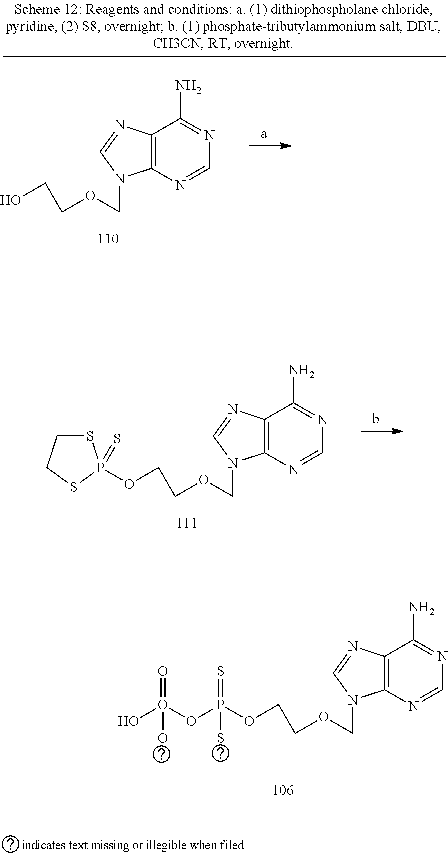

### Example 15: Synthesis of Adenine-Pα,α-dithiophosphate Analog

Adenine-Pα,α-dithiophosphate analogs 107, 108, and 109 were synthesized from thio-dithiaphospholane intermediate 111, via nucleophilic ring opening by: pyrophosphate/bisphosphonate/methylene-dichlorophosphonate, respectively, with DBU mediation (Scheme 13). The reactions were performed at RT for overnight with complete exclusion of moisture. Separation on an anion exchanger (elution with a linear gradient of ammonium bicarbonate solution) followed by HPLC purification gave 107, 108, and 109 in 21, 59, and 45% yield, respectively.

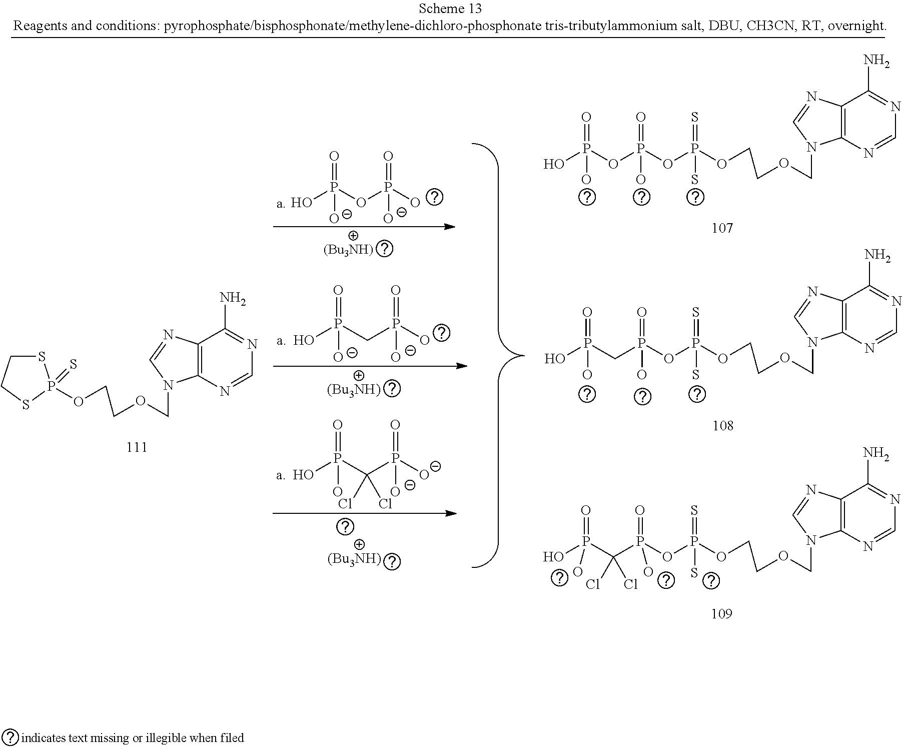

### Example 16: Screening Results for New Compounds at 10 μM vs. Human NPP1

TABLE 6
Screening results for new compounds at 10 μM vs. human NPP1.

| | | % inhibition ± SEM at 10 μM (n = 3) or | |
|---|---|---|---|
| | | (Kᵢ (μM ± SD)) | |
| | | p-Nph-5′-TMP (100 | |
| | | μM) as a | ATP (100 μM) |
| Comp. | Structure | substrate | as a substrate |
| 107 | 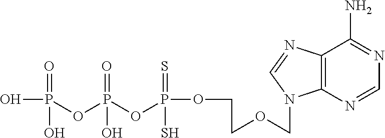 [SMILES: Nc1ncnc2c1ncn2COCCOP(=S)(S)OP(=O)(O)OP(=O)(O)O] | 74 ± 1 Kᵢ value: 1.98 ± 0.41 μM | 66 ± 1 Kᵢ value: 0.334 ± 0.550 μM |
| 108 | 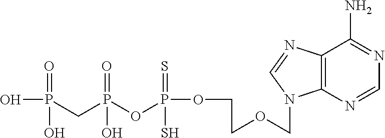 [SMILES: Nc1ncnc2c1ncn2COCCOP(=S)(S)OP(=O)(O)CP(=O)(O)O] | 65 ± 1 Kᵢ value: 2.16 ± 0.16 μM | 16 ± 3 |
| 109 | 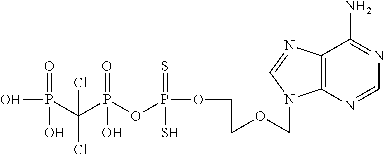 [SMILES: Nc1ncnc2c1ncn2COCCOP(=S)(S)OP(=O)(O)C(Cl)(Cl)P(=O)(O)O] | 85 ± 2 Kᵢ value: 0.698 ± 0.111 μM | 37 ± 1 |
| 106 | 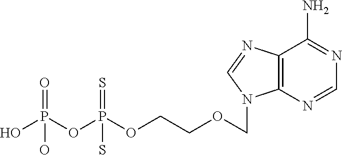 [SMILES: Nc1ncnc2c1ncn2COCCOP(=S)(S)OP(=O)(O)O] | 67 ± 1 Kᵢ value: 1.81 ± 0.41 μM | 68 ± 1 Kᵢ value: 0.305 ± 0.441 μM |

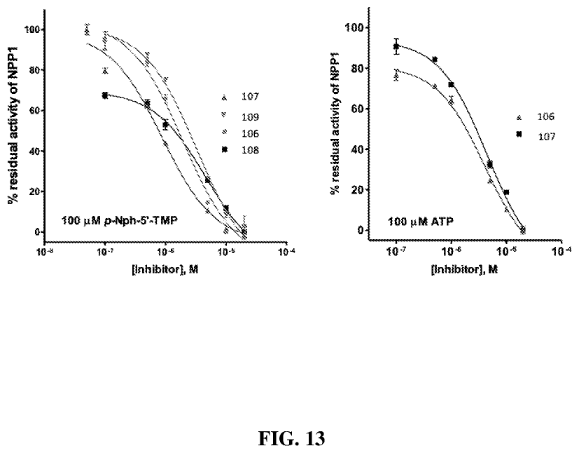

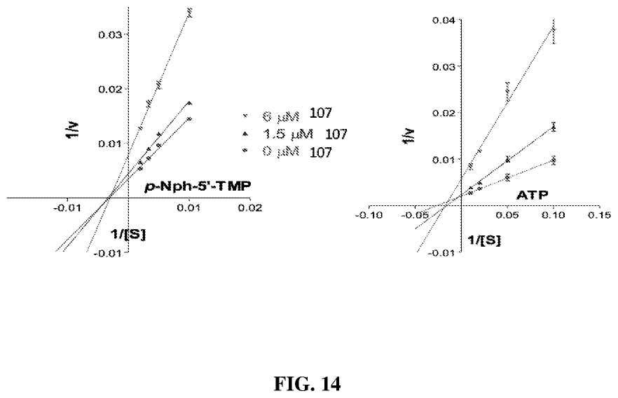

Reference is now made to FIG. 13 and FIG. 14, demonstrating Concentration-dependent inhibition curves for different inhibitors vs. p-Nph-5′-TMP and ATP as substrate (mean±SD of triplicate investigation) and inhibition type of analog 107 vs p-nph-5′-TMP and ATP respectively. Data generated by lineweaver-burk plot. α value of ˜1 shows non-competitive inhibition.

### Example 17: Selectivity Tests: Inhibitory Potency for Different Ecto-Nucleotidases

Analogues 106 and 107 were further tested vs. different ecto-nucleotidases including human NPP3, human CD39 and rat CD73 (Table 7). The results showed minute inhibition of other ecto-nucleotidases, indicating high selectivity for human NPP1.

TABLE 7
Selectivity tests with different ecto-nucleotidases.

| | % inhibition ± SEM (n = 3) | | |
|---|---|---|---|
| Compounds | human NPP3 ₐ | human CD39 _{b} | rat CD73 _{c} |
| 107 | 17 ± 1 | 28 ± 1 | 10 ± 4 |
| 106 | n.i._{d} | 14 ± 1 | n.i._{d} |

ₐ Investigation with 100 μM p-Nph-5′-TMP as a substrate
_{b} Investigation with 0.5 μM PSB-170621A as a substrate
_{c} Investigation with 5 μM [₃H]AMP as a substrate
_{d}No inhibition

### Example 18: Assessment for Agonist Potency on the Human P2Y1, P2Y2, P2Y4, and P2Y6 Receptor of the Acyclic Adenine-5′-Pα,α-Dithiophosphate Analogues

Given the structural resemblance of 106-109 to the natural ligands of P2Y receptors, adenine and uracil nucleotides as natural substrates, were assessed for agonist potency on the human P2Y1, P2Y2, P2Y4, and P2Y6 receptor. To evaluate NPP1 selectivity of analogs 106-109, they were tested for their ability to activate of the the uridine-nucleotide sensitive human recombinant P2Y1,2,4,6-receptors. The agonist potencies of this series of nucleotide analogues on the Gq protein-coupled human P2Y1R, P2Y2R, P2Y4R, and P2Y6R was determined by assessing its ability to induce calcium mobilization in stably transfected 1321N1 astrocytoma cells (Table 8). The nucleotide analogues were found in calcium mobilization assays to activate the P2Y2R with mid-micromolar potency. All assessed compounds were inactive at the P2Y1R, P2Y4R, and P2Y6R at 10 μM.

TABLE 8
Potency of nucleotide analogues at various human P2YRs.

| | % activation at 10 μMₐ | | | |
|---|---|---|---|---|
| Compound | P2Y₁ | P2Y₄ | P2Y₆ | P2Y2 |
| 107 | −1 ± 1 | −2 ± 2 | −5 ± 3 | 39 ± 13 |
| 109 | −2 ± 1 | 0 ± 2 | 2 ± 4 | 21 ± 5 |
| 106 | 2 ± 2 | 0 ± 0 | −2 ± 1 | 32 ± 18 |
| 108 | 0 ± 1 | −2 ± 1 | −8 ± 2 | 44 ± 9 |

ₐPotency to elicit calcium mobilization in 1321N1 astrocytoma cells recombinantly expressing human P2YRs.
Shown are the mean values of 2 independent experiments, each performed in duplicate.

### Example 19: Assessment of Effects on TNAP Activity on Primary Human Chondrocytes

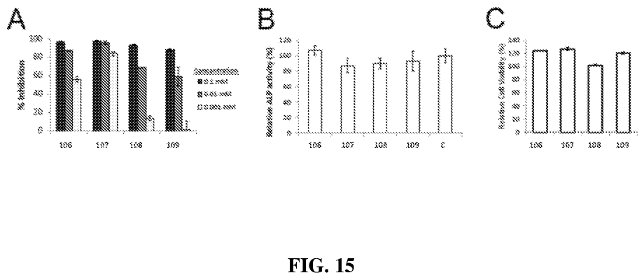

To confirm that the analogs 106-109 do not interfere with tissue non-specific alkaline phosphatase (TNAP) activity in adult human cartilage, we tested their effects on TNAP activity on primary human chondrocytes. None of the analogs displayed any significant inhibition of TNAP activity relative to untreated chondrocytes as demonstrated on FIG. 15A-B. It was also confirmed that the analogues were non-toxic to cultured chondrocytes. Indeed, according to the XTT assay none of the analogs were detrimental to cell viability, and interestingly, analogs 106, 107 and 109 appeared to slightly increase cell survival as demonstrated on FIG. 15C.

Discussion:

Analogue 106 (ADP derivatives) and 107 (ATP derivative) are the most potent inhibitor of human NPP1 vs. p-Nph-5′-TMP and ATP as a substrate with low μM IC50 values. Analogue 108 and 109 were also ATP analogues however, oxygen bridge between β-γ-phosphates in analogue 106 were replaced by either —CH₂— 108 and —CCl2 109. This modification leads to reduction in the inhibitory potency. Lower IC50 values were observed for analogue 107 vs. p-Nph-5′-TMP and ATP as a substrate.

### REFERENCES

1. Bollen, M.; Gijsbers, R.; Ceulemans, H.; Stalmans, W.; Stefan, C. Nucleotide pyrophosphatases/phosphodiesterases on the move. Crit. Rev. Biochem. Mol. Biol. 2000, 35, 393-432.
2. Yegutkin, G. G. Nucleotide- and nucleoside-converting ectoenzymes: important modulators of purinergic signalling cascade. Biochim. Biophys. Acta. 2008, 1783, 673-694.
3. Albright, R. A.; Ornstein, D. L.; Cao, W.; Chang, W. C.; Robert, D.; Tehan, M.; Hoyer, D.; Liu, L.; Stabach, P.; Yang, G. Molecular basis of purinergic signal metabolism by ectonucleotide pyrophosphatase/phosphodiesterases 4 and 1 and implications in stroke. J. Biol. Chem. 2014, 289, 3294-3306.
4. Freeman, R. V.; Otto, C. M. Spectrum of calcific aortic valve disease. Circulation 2005, 111, 3316-3326.
5. El Husseini, D.; Boulanger, M.-C.; Mahmut, A.; Bouchareb, R.; Laflamme, M.-H.; Fournier, D.; Pibarot, P.; Bosse, Y.; Mathieu, P. P2Y2 receptor represses IL-6 expression by valve interstitial cells through Akt: implication for calcific aortic valve disease. J. mol. cell. cardiol. 2014, 72, 146-156.
6. Cote, N. E. H., D.; Pepin, A.; Celine B.; Gilbert, L. A.; Audet, A.; Fournier, D.; Pibarot, P.; Moreau, P.; Mathieu, M. Inhibition of ectonucleotidase with ARL67156 prevents the development of calcific aortic valve disease in warfarin-treated rats. Eur. J. Pharmacol. 2012, 689, 139-146.
7. Hashimoto, S.; Ochs, R. L.; Rosen, F.; Quach, J.; McCabe, G.; Solan, J.; Seegmiller, J. E.; Terkeltaub, R.; Lotz, M. Chondrocyte-derived apoptotic bodies and calcification of articular cartilage. Proc. Natl Acad. Sci. 1998, 95, 3094-3099.
8. Johnson, K.; Hashimoto, S.; Lotz, M.; Pritzker, K.; Goding, J.; Terkeltaub, R. Up-regulated expression of the phosphodiesterase nucleotide pyrophosphatase family member PC-1 is a marker and pathogenic factor for knee meniscal cartilage matrix calcification. Arthritis Rheum. 2001, 44, 1071-1081.
9. Pattrick, M.; Hamilton, E.; Hornby, J.; Doherty, M. Synovial fluid pyrophosphate and nucleoside triphosphate pyrophosphatase: comparison between normal and diseased and between inflamed and non-inflamed joints. Ann. Rheum. Dis. 1991, 50, 214-218.
10. Stefan, C.; Jansen, S.; Bollen, M. NPP-type ectophosphodiesterases: unity in diversity. Trends Biochem. Sci. 2005, 30, 542-550.
11. Shayhidin, E. E.; Forcellini, E.; Boulanger, M. C.; Mahmut, A.; Dautrey, S.; Barbeau, X.; Lagüe, P.; Sevigny, J.; Paquin, J. F.; Mathieu, P. Quinazoline-4-piperidine sulfamides are specific inhibitors of human NPP1 and prevent pathological mineralization of valve interstitial cells. Br. J. Pharmacol. 2015, 172, 4189-4199.
12. Ye, Y.-M.; Yang, E.-M.; Yoo, H.-S.; Shin, Y.-S.; Kim, S.-H.; Park, H.-S. Increased level of basophil CD203c expression predicts severe chronic urticaria. J. Korean Med. Sci. 2014, 29, 43-47.
13. Goding, J. W.; Grobben, B.; Slegers, H. Physiological and pathophysiological functions of the ecto-nucleotide pyrophosphatase/phosphodiesterase family. Biochim. Biophys. Acta. 2003, 1638, 1-19.
14. Iung, B.; Baron, G.; Butchart, E. G.; Delahaye, F.; Gohlke-Barwolf, C.; Levang, O. W.; Tornos, P.; Vanoverschelde, J.-L.; Vermeer, F.; Boersma, E. A prospective survey of patients with valvular heart disease in Europe: The Euro Heart Survey on Valvular Heart Disease. Eur. Heart J. 2003, 24, 1231-1243.
15. Nadel, Y.; Lecka, J.; Gilad, Y.; Ben-David, G.; Farster, D.; Reiser, G.; Kenigsberg, S.; Camden, J.; Weisman, G. A.; Senderowitz, H. Highly Potent and Selective Ectonucleotide Pyrophosphatase/Phosphodiesterase I Inhibitors Based on an Adenosine 5′-(α or γ)-Thio-(α, β- or β, γ)-methylenetriphosphate Scaffold. J. Med. Chem. 2014, 57, 4677-4691.
16. Rajamannan, N. M. Calcific aortic stenosis: lessons learned from experimental and clinical studies. Arterioscler. Thromb. Vasc. Biol. 2009, 29, 162.
17. Orriss, I. R.; Utting, J. C.; Brandao-Burch, A.; Colston, K.; Grubb, B. R.; Burnstock, G.; Arnett, T. R. Extracellular Nucleotides Block Bone Mineralization in Vitro: Evidence for Dual Inhibitory Mechanisms Involving Both P2Y2 Receptors and Pyrophosphate. Endocrinology 2007, 148, 4208-4216.
18. Côté, N.; El Husseini, D.; Pépin, A.; Guauque-Olarte, S.; Ducharme, V.; Bouchard-Cannon, P.; Audet, A.; Fournier, D.; Gaudreault, N.; Derbali, H.; McKee, M. D.; Simard, C.; Després, J.-P.; Pibarot, P.; Bossé, Y.; Mathieu, P. ATP acts as a survival signal and prevents the mineralization of aortic valve. J. Mol. Cell. Cardiol. 2012, 52, 1191-1202.
19. Qian, S.; Regan, J. N.; Shelton, M. T.; Hoggatt, A.; Mohammad, K. S.; Herring, P. B.; Seye, C. I. The P2Y 2 nucleotide receptor is an inhibitor of vascular calcification. Atherosclerosis 2017, 257, 38-46.
20. Eliahu, S. E.; Camden, J.; Lecka, J.; Weisman, G. A.; Sevigny, J.; Gelinas, S.; Fischer, B. Identification of hydrolytically stable and selective P2Y1 receptor agonists. Eur. J. Med. Chem. 2009, 44, 1525-1536.
21. Eliahu, S.; Lecka, J.; Reiser, G.; Haas, M.; Bigonnesse, F.; Levesque, S. A.; Pelletier, J.; Sevigny, J.; Fischer, B. Diadenosine 5′,5″-(Boranated)polyphosphonate Analogues as Selective Nucleotide Pyrophosphatase/Phosphodiesterase Inhibitors. J. Med. Chem. 2010, 53, 8485-8497.
22. Khan, K. M.; Fatima, N.; Rasheed, M.; Jalil, S.; Ambreen, N.; Perveen, S.; Choudhary, M. I. 1,3,4-Oxadiazole-2(3H)-thione and its analogues: A new class of non-competitive nucleotide pyrophosphatases/phosphodiesterases 1 inhibitors. Bioorg. Med. Chem. 2009, 17, 7816-7822.
23. Patel, S. D.; Habeski, W. M.; Cheng, A. C.; de la Cruz, E.; Loh, C.; Kablaoui, N. M. Quinazolin-4-piperidin-4-methyl sulfamide PC-1 inhibitors: Alleviating hERG interactions through structure-based design. Bioorg. Med. Chem. Lett 2010, 20, 2685-2685.
24. Azran, S.; Danino, O.; Farster, D.; Kenigsberg, S.; Reiser, G.; Dixit, M.; Singh, V.; Major, D. T.; Fischer, B. Identification of Highly Promising Antioxidants/Neuroprotectants Based on Nucleoside 5′-Phosphorothioate Scaffold. Synthesis, Activity, and Mechanisms of Action. J. Med. Chem. 2015, 58, 8427-8443.
25. Jacobson, K. A.; Boeynaems, J. M. P2Y nucleotide receptors: promise of therapeutic applications. Drug Discov. Today 2010, 15, 570-578.
26. Jacobson, K. A.; Costanzi, S.; Ivanov, A. A.; Tchilibon, S.; Besada, P.; Gao, Z.-G.; Maddileti, S.; Harden, T. K. Structure activity and molecular modeling analyses of ribose- and base-modified uridine 5′-triphosphate analogues at the human P2Y2 and P2Y4 receptors. Biochem. Pharmacol. 2006, 71, 540-549.
27. Frey, P. A.; Sammons, R. D. Bond order and charge localization in nucleoside phosphorothioates. Science 1985, 228, 541-5.
28. Sigel, R. K. O.; Song, B.; Sigel, H. Stabilities and Structures of Metal Ion Complexes of Adenosine 5′-O-Thiomonophosphate (AMPS2-) in Comparison with Those of Its Parent Nucleotide (AMP2-) in Aqueous Solution. J. Am. Chem. Soc 1997, 119, 744-755.
29. Sayer, A. H.; Itzhakov, Y.; Stern, N.; Nadel, Y.; Fischer, B. Characterization of Complexes of Nucleoside-5′-Phosphorothioate Analogues with Zinc Ions. Inorg. Chem. 2013, 52, 10886-10896.
30. Okruszek, A.; Olesiak, M.; Balzarini, J. The Synthesis of Nucleoside 5′-O-(1, 1-Dithiotriphosphates). J. Med. Chem. 1994, 37, 3850-3854.
31. von Kügelgen, I.; Hoffmann, K. Pharmacology and structure of P2Y receptors. Neuropharmacology 2016, 104, 50-61.
32. Trinquet, E.; Bouhelal, R.; Dietz, M. Monitoring Gq-coupled receptor response through inositol phosphate quantification with the IP-One assay. Expert Opin. Drug Discovery. 2011, 6, 981-994.
33. Lecka, J.; Ben-David, G.; Simhaev, L.; Eliahu, S.; Oscar Jr, J.; Luyindula, P.; Pelletier, J.; Fischer, B.; Senderowitz, H.; Sevigny, J. Nonhydrolyzable ATP analogues as selective inhibitors of human NPP1: a combined computational/experimental study. J. Med. Chem. 2013, 56, 8308-8320.
34. Hillaire-Buys, D.; Shahar, L.; Fischer, B.; Chulkin, A.; Linck, N.; Chapal, J.; Loubatieres-Mariani, M. M.; Petit, P. Pharmacological evaluation and chemical stability of 2-benzylthioether-5′-O-(1-thiotriphosphate)-adenosine, a new insulin secretagogue acting through P2Y receptors. Drug Dev. Res. 2001, 53, 33-43.
35. Eliahu, S.; Lecka, J.; Reiser, G.; Bigonnesse, F.; Sevigny, J.; Fischer., B. Diadenosine Poly(borano)phosphate analogues as specific NPP inhibitors. J. Med. Chem. 2010, 53, 8485-8497.
36. Baykov, A.; Evtushenko, O.; Avaeva, S. A malachite green procedure for orthophosphate determination and its use in alkaline phosphatase-based enzyme immunoassay. Anal. biochem. 1988, 171, 266-270.
37. Meltzer, D.; Ethan, O.; Arguin, G.; Nadel, Y.; Danino, O.; Lecka, J.; Sevigny, J.; Gendron, F.-P.; Fischer, B. Synthesis and structure-activity relationship of uracil nucleotide derivatives towards the identification of human P2Y 6 receptor antagonists. Bioorg. Med. Chem. 2015, 23, 5764-5773.

1. A Compound having the structure, 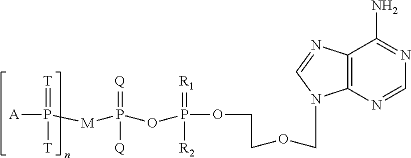
or a pharmaceutically acceptable salt thereof, wherein:
R₁ and R₂ is each independently selected from O or S;
Q is selected from O or S;
M is selected from O, OH, CH₂, CCl₂, CBr₂, and CF₂;
T is selected from O or S;
A is selected from O—, OH, and a nucleoside; and,
n is 0 or 1.

2. The compound of claim 1, wherein both R₁ and R₂ are S; Q is O; M is CH₂; T is O; n is 1; and A is O.

3. The compound of claim 1, wherein both R₁ and R₂ are S; Q is O; M is O; T is O; n is 1; and A is OH.

4. The compound of claim 1, wherein both R₁ and R₂ are S; Q is O; M is O; T is O; and n is 0.

5. The compound of claim 1, wherein both R₁ and R₂ are S; Q is O; M is CCl₂; T is O; and n is 1; and A is O.

6. The Compound of claim 1, having the structure: 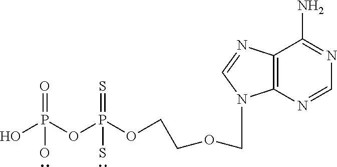 [SMILES: Nc1ncnc2c1ncn2COCCOP(=S)(S)OP(=O)(O)O]
or a pharmaceutically acceptable salt thereof.

7. The Compound of claim 1, having the structure: 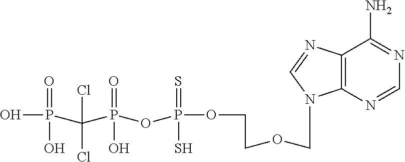 [SMILES: Nc1ncnc2c1ncn2COCCOP(=S)(S)OP(=O)(O)C(Cl)(Cl)P(=O)(O)O]
or a pharmaceutically acceptable salt thereof.

8. The Compound of claim 1, having the structure: 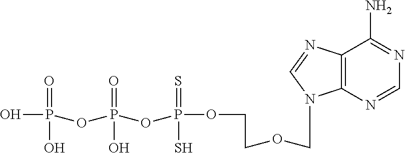 [SMILES: Nc1ncnc2c1ncn2COCCOP(=S)(S)OP(=O)(O)OP(=O)(O)O]
or a pharmaceutically acceptable salt thereof.

9. The Compound of claim 1, having the structure: 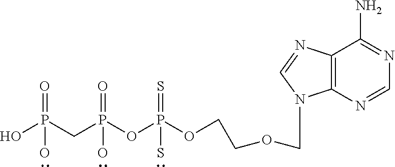 [SMILES: Nc1ncnc2c1ncn2COCCOP(=S)(S)OP(=O)(O)CP(=O)(O)O]
or a pharmaceutically acceptable salt thereof.

10. A pharmaceutical composition comprising a Compound having the structure: 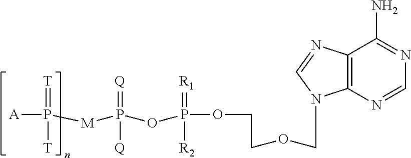
or a pharmaceutically acceptable salt thereof, wherein:
R₁ and R₂ is each independently selected from O and S;
Q is selected from O and S;
M is selected from O, OH, CH₂, CCl₂, CBr₂, and CF₂;
T is selected from O and S;
A is selected from O—, OH, and a nucleoside;
n is 0 or 1;
and at least one pharmaceutically acceptable carrier.

11. The pharmaceutical composition of claim 10, wherein:
a. both R₁ and R₂ are S; Q is O; M is CH₂; T is O; n is 1; and A is O;
b. both R₁ and R₂ are S; Q is O; M is O; T is O; n is 1; and A is OH;
c. both R₁ and R₂ are S; Q is O; M is O; T is O; and n is 0; or,
d. both R₁ and R₂ are S; Q is O; M is CCl₂; T is O; and n is 1; and A is O.

12. (canceled)

13. (canceled)

14. (canceled)

15. The pharmaceutical composition of claim 10, comprising a Compound having the structure: 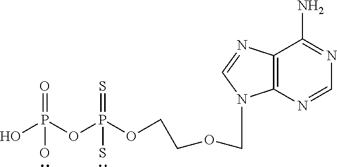 [SMILES: Nc1ncnc2c1ncn2COCCOP(=S)(S)OP(=O)(O)O]
or a pharmaceutically acceptable salt thereof, and at least one pharmaceutically acceptable carrier.

16. The pharmaceutical composition of claim 10, comprising a Compound having the structure: 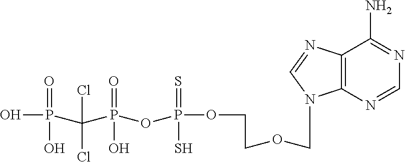 [SMILES: Nc1ncnc2c1ncn2COCCOP(=S)(S)OP(=O)(O)C(Cl)(Cl)P(=O)(O)O]
or a pharmaceutically acceptable salt thereof, and at least one pharmaceutically acceptable carrier.

17. The pharmaceutical composition of claim 1, comprising a Compound having the structure: 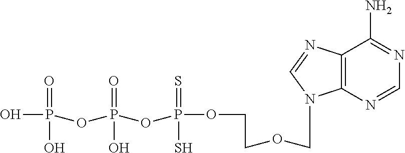 [SMILES: Nc1ncnc2c1ncn2COCCOP(=S)(S)OP(=O)(O)OP(=O)(O)O]
or a pharmaceutically acceptable salt thereof, and at least one pharmaceutically acceptable carrier.

18. The pharmaceutical composition of claim 10, comprising a Compound having the structure: 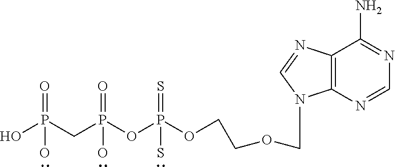 [SMILES: Nc1ncnc2c1ncn2COCCOP(=S)(S)OP(=O)(O)CP(=O)(O)O]
or a pharmaceutically acceptable salt thereof, and at least one pharmaceutically acceptable carrier.

19. A method for treating a condition associated with enhanced NNPI activity in a subject in need of such treatment comprising administering to the subject a therapeutically effective amount of the pharmaceutical composition claim 10.

20. A method for treating a condition associated with elevated NNP1 levels in a subject in need of such treatment, comprising administering to the subject a therapeutically effective amount of the pharmaceutical composition claim 10.

21. The method of claim 20, wherein the condition associated with elevated levels of NNP1 is calcific aortic valve disease (CAVD) or Calcium Pyrophosphate Dihydrate (CPPD) disease.

22. (canceled)

23. (canceled)

24. (canceled)

25. (canceled)
